(19) United States
(12) Patent Application Publication
(10) Pub. No.: US 2016/0067120 A1
(43) Pub. Date: Mar. 10, 2016

(54) ABSORBENT ARTICLES AND METHODS AND SYSTEMS OF PACKAGING THEM

(71) Applicants: E. Wayne Meeks, Collinsville, VA (US); Peter J. Gawley, Roanoke, VA (US); Robert A. Baldacci, Roanoke, VA (US); Ronald G. McCollum, Martinsville, VA (US)

(72) Inventors: E. Wayne Meeks, Collinsville, VA (US); Peter J. Gawley, Roanoke, VA (US); Robert A. Baldacci, Roanoke, VA (US); Ronald G. McCollum, Martinsville, VA (US)

(21) Appl. No.: 14/800,921

(22) Filed: Jul. 16, 2015

Related U.S. Application Data
(60) Provisional application No. 62/025,835, filed on Jul. 17, 2014.
(60) Provisional application No. 62/026,209, filed on Jul. 18, 2014.

(51) Int. Cl. A61F 13/551; B65B 5/06; B65B 5/10; B65B 61/20; B65B 25/14

(52) U.S. Cl. CPC A61F 13/551; B65B 61/207; B65B 25/14; B65B 5/10; B65B 5/06

(57) ABSTRACT

Certain configurations of articles comprising stacked layers of absorbent articles are described herein. In certain examples, the absorbent articles of a particular layer are arranged in two or more lanes. In some instances, adjacent stacks of the stacked layers are separated by a divider configured to provide support to at least one lane of absorbent articles of a first stack. Systems and methods of producing and packaging the articles are also described.

### PRIORITY APPLICATIONS

This application claims priority to, and the benefit of, each of U.S. Provisional Application No. 62/025,835 filed on Jul. 17, 2014 and to U.S. Provisional Application No. 62/026,209 filed on Jul. 18, 2014, the entire disclosure of each of which is hereby incorporated herein by reference for all purposes.

### TECHNOLOGICAL FIELD

Certain features, aspects and embodiments are directed to absorbent articles. More particularly, certain configurations described herein are directed to two or more layered stacks of absorbent articles packaged with a divider.

### BACKGROUND

Absorbent articles can be used for many different purposes. In some instances, absorbent articles can be used as hygiene articles or for other commercial and personal uses.

### SUMMARY

Certain aspects described herein are directed to articles comprising two or more layered stacks of absorbent articles packaged with a divider. In some instances, each layer of a particular stack may comprise a plurality of individual absorbent articles. In some configurations, the divider can be configured to provide support to one or more lanes of a particular stack to deter, for example, collapse of one or more lanes of the stack. Illustrative configurations of the dividers and systems and methods of packaging the articles with a divider are described in more detail below.

In one aspect, an article comprising a first stack comprising a plurality of individual absorbent articles, in which the absorbent articles of the first stack are present in stacked layers with each layer of the first stack comprising a plurality of the individual, absorbent articles arranged in lanes, a second stack adjacent to the first stack, in which the second stack comprises a plurality of individual absorbent articles, in which the absorbent articles of the second stack are present in stacked layers with each layer of the second stack comprising a plurality of the individual, absorbent articles arranged in lanes, and a divider between at least some portion of the first stack and the second stack, the divider sized and arranged to provide support to at least one lane of absorbent articles of the first stack is provided.

In certain embodiments, a terminal layer of the first stack is coupled to a first layer of the second stack. In other embodiments, the divider is sized and arranged as one of a U-shaped member, a Z-shaped member, a C-shaped member, a lattice member or an X-shaped member. In some examples, the divider comprises a continuous, unitary member. In further examples, the divider comprises one or more of a paper, a plastic, a metal, or combinations thereof. In some instances, the divider is configured as a laminate. In other examples, the divider is sized and arranged to comprise a height less than a height of the first stack. In certain configurations, the divider is sized and arranged to comprise a width substantially the same as the width of the first stack. In other configurations, the divider is sized and arranged to comprise a height less than a full height of the first stack. In some embodiments, the divider is sized and arranged to provide support to an upper portion of the first stack. In some examples, a height of the divider is variable. In further instances, each layer of the first stack comprises at least three separate lanes on each layer of the first stack. In some embodiments, each layer of the first stack comprises at least four separate lanes on each layer of the first stack. In some instances, each layer of the second stack comprises at least four separate lanes on each layer of the second stack. In certain examples, a terminal layer of the first stack is coupled to a first layer of the second stack. In other examples, each lane of each layer of the first stack is separated from other lanes in each layer of the first stack through one or more perforations. In some embodiments, the divider is sized and arranged to provide support to a terminal lane of the first stack. In further examples, the divider comprises at least one projection configured to engage the terminal lane of the first stack. In some examples, the divider comprises a plurality of projections each configured to engage a respective lane of the first stack. In certain examples, the divider comprises a movable component configured to engage a first lane of the first stack in a first position of the movable component and to move to a second position to engage a second lane of the first stack, adjacent to the first lane of the first stack, after all absorbent articles in the first lane of the first stack are removed from the first stack.

In another aspect, an article comprising a first stack comprising a plurality of individual absorbent articles each comprising an absorbent material layer disposed on a substrate layer, in which the absorbent articles of the first stack are present in stacked layers with each layer of the first stack comprising a plurality of the individual, absorbent articles arranged in a plurality of lanes with adjacent lanes separated by perforations in the substrate layer, a second stack adjacent to the first stack, in which the second stack comprises a plurality of individual absorbent articles each comprising an absorbent material layer disposed on a substrate layer, in which the absorbent articles of the second stack are present in stacked layers with each layer of the second stack comprising a plurality of the individual, absorbent articles arranged in a plurality of lanes with adjacent lanes separated by perforations in the substrate layer, and wherein a layer of the first stack is coupled to a layer of the second stack through the substrate layer, and a divider between at least some portion of the first stack and the second stack, the divider sized and arranged to provide support to at least one lane of absorbent articles of the first stack is disclosed.

In certain embodiments, the article comprises a third stack adjacent to the second stack, in which the third stack comprises a plurality of individual absorbent articles each comprising an absorbent material layer disposed on a substrate layer, in which the absorbent articles of the third stack are present in stacked layers with each layer of the third stack comprising a plurality of the individual, absorbent articles arranged in a plurality of lanes with adjacent lanes separated by perforations in the substrate layer, and wherein a layer of the second stack is coupled to a layer of the third stack through the substrate layer. In some instances, the divider is sized and arranged as one of a U-shaped member, a Z-shaped member, a C-shaped member, a lattice member or an X-shaped member. In other instances, the divider comprises a continuous, unitary member. In some examples, the divider comprises one or more of a paper, a plastic, a metal, or combinations thereof. In further examples, the divider is configured as a laminate. In some examples, the divider is sized and arranged to comprise a height less than a height of the first stack. In additional examples, the divider is sized and arranged to comprise a width substantially the same as the width of the first stack. In some embodiments, the divider is sized and arranged to comprise a height less than a full height of the first stack. In certain examples, the divider is sized and arranged to provide support to an upper portion of the first stack. In other examples, a height of the divider is variable. In some examples, each layer of the first stack comprises at least three separate lanes on each layer of the first stack. In some embodiments, each layer of the first stack comprises at least four separate lanes on each layer of the first stack. In other embodiments, each layer of the second stack comprises at least four separate lanes on each layer of the second stack. In certain examples, a terminal layer of the first stack is coupled to a first layer of the second stack. In some examples, each lane of each layer of the first stack is separated from other lanes in each layer of the first stack through one or more perforations. In other examples, the divider is sized and arranged to provide support to a terminal lane of the first stack. In some embodiments, the divider comprises at least one projection configured to engage the terminal lane of the first stack. In certain instances, the divider comprises a plurality of projections each configured to engage a respective lane of the first stack. In other instances, the divider comprises a movable component configured to engage a first lane of the first stack in a first position of the movable component and to move to a second position to engage a second lane of the first stack, adjacent to the first lane of the first stack, after all absorbent articles in the first lane of the first stack are removed from the first stack.

In an additional aspect, an article configured to receive a plurality of absorbent articles, the article comprising a container sized and arranged to receive at least two stacks each comprising the plurality of the absorbent articles, in which each of the stacks comprises a plurality of layers with each layer comprising a plurality of the individual, absorbent articles arranged in lanes, and a divider configured to be inserted into the container, the divider configured to be positioned between the at least two stacks when inserted into the container, wherein the divider is sized and arranged to provide support to at least one lane of absorbent articles of one of the stacks is described.

In certain examples, the container comprises a lower component configured to receive the at least two stacks and the divider and an upper component configured to removably couple to the lower component and configured to slide over the stacks when coupled to the lower component. In other examples, the divider is integral to the upper component of the container. In some embodiments, the divider is integral to the lower component. In additional embodiments, the divider is sized and arranged as one of a U-shaped member, a Z-shaped member, a C-shaped member, a lattice member or an X-shaped member. In further examples, the divider comprises a continuous, unitary member. In additional examples, the divider comprises one or more of a paper, a plastic, a metal, or combinations thereof. In some embodiments, the divider is configured as a laminate. In other embodiments, the divider is sized and arranged to comprise a height less than a height of the container. In further examples, the divider comprises a plurality of sections in which one of the sections is sized and arranged to bisect the container. In some examples, a height of the divider is variable. In other embodiments, the container comprises a slope at a bottom surface. In certain embodiments, the article comprises an insert configured to provide a slope to a bottom surface of the container when inserted into the container. In some embodiments, the divider is sized and arranged to provide support to a terminal lane of one of the stacks. In certain examples, the divider comprises at least one projection configured to engage the terminal lane of the one of the stacks. In other examples, the divider comprises a plurality of projections each configured to engage a respective lane of one of the stacks. In some examples, the divider comprises a movable component configured to engage a first lane of a first stack in a first position of the movable component and to move to a second position to engage a second lane of the first stack, adjacent to the first lane of the first stack, after all absorbent articles in the first lane of the first stack are removed from the first stack. In certain instances, the moveable component comprises a spring-loaded device. In other configurations, the moveable component is coupled to a stationary component of the divider through a slot in the stationary component. In some examples, the moveable component is configured to move toward a terminal lane of the first stack in a longitudinal direction of the slot.

In another aspect, an article configured to receive a plurality of absorbent articles, the article comprising a container sized and arranged to receive at least two stacks each comprising the plurality of the absorbent articles, a first stack in the container and comprising a plurality of individual absorbent articles, in which the absorbent articles of the first stack are present in stacked layers with each layer of the first stack comprising a plurality of the individual, absorbent articles arranged in lanes, and a divider configured to be inserted into the container, the divider configured to be positioned against the first stack when inserted into the container, wherein the divider is sized and arranged to provide support to at least one lane of absorbent articles of the first stack is disclosed.

In some configurations, the container comprises a lower component configured to receive the at least two stacks and the divider and an upper component configured to removably couple to the lower component and configured to slide over the stacks when coupled to the lower component. In other configurations, the divider is integral to the upper component of the container. In some examples, the divider is integral to the lower component. In further embodiments, the divider is sized and arranged as one of a U-shaped member, a Z-shaped member, a C-shaped member, a lattice member or an X-shaped member. In additional embodiments, the divider comprises a continuous, unitary member. In some examples, the divider comprises one or more of a paper, a plastic, a metal, or combinations thereof. In other examples, the divider is configured as a laminate. In certain configurations, the divider is sized and arranged to comprise a height less than a height of the first stack. In other configurations, the divider comprises a plurality of sections in which one of the sections is sized and arranged to bisect the container. In some embodiments, a height of the divider is variable. In other embodiments, the container comprises a slope at a bottom surface, in which the slope is present at an effective angle to tilt the first stack toward the divider. In certain examples, the article comprises an insert configured to provide a slope to a bottom surface of the container when inserted into the container, in which the slope is present at an effective angle to tilt the first stack toward the divider. In other examples, the divider is sized and arranged to provide support to a terminal lane of the first stack. In some embodiments, the divider comprises at least one projection configured to engage the terminal lane of the first stack. In certain instances, the divider comprises a plurality of projections each configured to engage a respective lane of the first stack. In some examples, the divider comprises a movable component configured to engage a first lane of a first stack in a first position of the movable component and to move to a second position to engage a second lane of the first stack, adjacent to the first lane of the first stack, after all absorbent articles in the first lane of the first stack are removed from the first stack. In other embodiments, the moveable component comprises a spring-loaded device. In some embodiments, the moveable component is coupled to a stationary component of the divider through a slot in the stationary component. In certain embodiments, the moveable component is configured to move toward a terminal lane of the first stack in a longitudinal direction of the slot.

In an additional aspect, a divider comprising a plurality of sections coupled to each other, in which the sections together are configured to position the divider between at least some portion of a first stack of a plurality of absorbent articles and a second stack of a plurality of absorbent articles, the divider sized and arranged to provide support to at least one lane of plurality of absorbent articles of the first stack is provided.

In certain examples, the divider is configured with at least three sections with at least two of the sections being foldable. In some examples, the divider is configured with at least three sections with at least two of the sections being coupled to each other through a hinge. In other examples, the divider is sized and arranged as one of a U-shaped member, a Z-shaped member, a C-shaped member, a lattice member or an X-shaped member. In some examples, the divider comprises a continuous, unitary member. In certain configurations, the divider comprises one or more of a paper, a plastic, a metal, or combinations thereof. In other configurations, the divider is configured as a laminate. In some embodiments, the divider is sized and arranged to comprise a height less than a height of the first stack. In certain examples, the divider is sized and arranged to comprise a width substantially the same as the width of the first stack. In some embodiments, the divider is sized and arranged to comprise a height less than a full height of the first stack. In certain examples, the divider is sized and arranged to provide support to an upper portion of the first stack. In other embodiments, a height of the divider is variable. In other examples, the divider is configured as a multi-layer laminate. In some examples, the divider comprises a coating on a surface of the divider. In certain embodiments, the coating comprises at least one of an antibacterial coating, an antifungal coating, an anti-static coating, a friction reducing coating and a hydrophobic coating. In other embodiments, the divider comprises at least one indicator. In certain configurations, the divider is sized and arranged to provide support to a terminal lane of the first stack. In some examples, the divider comprises at least one projection configured to engage the terminal lane of the first stack. In other examples, the divider comprises a plurality of projections each configured to engage a respective lane of the first stack. In certain instances, the divider comprises a movable component configured to engage a first lane of the first stack in a first position of the movable component and to move to a second position to engage a second lane of the first stack, adjacent to the first lane of the first stack, after all absorbent articles in the first lane of the first stack are removed from the first stack.

In another aspect, a method of packaging a plurality of absorbent articles comprising stacking a first stack comprising a plurality of absorbent articles in a container, in which the absorbent articles of the first stack are arranged in lanes in the first stack, stacking a second stack adjacent to the stacked first stack in the container, the second stack comprising a plurality of absorbent articles, in which the absorbent articles of the second stack are arranged in lanes in the second stack, and inserting a divider between the first stack and the second stack, in which the divider is sized and arranged to provide support to at least one lane of absorbent articles of the first stack.

In certain configurations, the method comprises inserting the divider into the container after the first stack is stacked and before the second stack is fully stacked. In other configurations, the method comprises inserting the divider into the container before either of the first stack and the second stack is fully stacked. In some instances, the method comprises configuring the divider to comprise a U-shaped member, a Z-shaped member, a C-shaped member, a lattice member or an X-shaped member. In additional examples, the method comprises coupling a bottom layer of the first stack to a top layer of the second stack. In further embodiments, the method comprises coupling a bottom layer of the first stack to a bottom layer of the second stack. In some instances, the method comprises inserting a first divider and a second divider between the first stack and the second stack. In certain embodiments, the method comprises configuring each of the first stack and the second stack with at least 4 lanes of absorbent articles per layer. In some examples, the method comprises covering the container with another container after insertion of the divider. In some embodiments, the method comprises covering the container with another container comprising the divider.

In an additional aspect, a method of providing support to a stack of absorbent articles comprising inserting a divider between a first stack of absorbent articles and a second stack of absorbent articles each positioned in a container, in which each of the first stack and the second stack comprises a plurality of individual absorbent articles, in which the absorbent articles of the first stack are present in stacked layers with each layer of the first stack comprising a plurality of the individual, absorbent articles arranged in lanes is disclosed.

In certain configurations, the method comprises inserting the divider into the container after the first stack is stacked and before the second stack is fully stacked. In other configurations, the method comprises inserting the divider into the container before either of the first stack and the second stack is fully stacked. In additional configurations, the method comprises configuring the divider to comprise a U-shaped member, a Z-shaped member, a C-shaped member, a lattice member or an X-shaped member. In some examples, the method comprises coupling a bottom layer of the first stack to a top layer of the second stack. In certain configurations, the method comprises coupling a bottom layer of the first stack to a bottom layer of the second stack. In other configurations, the method comprises inserting a first divider and a second divider between the first stack and the second stack. In further examples, the method comprises configuring each of the first stack and the second stack with at least four lanes of absorbent articles per layer. In other examples, the method comprises covering the container with another container after insertion of the divider. In certain embodiments, the method comprises covering the container with another container comprising the divider.

In another aspect, a method of producing an individual absorbent article comprising removing at least one lane of absorbent articles from a container comprising a first stack of absorbent articles, a second stack of absorbent articles and a divider between the first stack and the second stack, in which each layer of the first stack and the second stack comprises a plurality of lanes of absorbent articles coupled to each other, and decoupling an absorbent article in the removed lane from other absorbent articles in the removed lane to provide the individual absorbent article is provided.

In some configurations, the method comprises configuring the divider to move toward a remaining lane of the first stack after another lane is removed from the first stack. In other examples, the method comprises configuring the divider to comprise a U-shaped member, a Z-shaped member, a C-shaped member, a lattice member or an X-shaped member. In further instances, the method comprises configuring the lanes with perforations to separate the lanes from each other. In some examples, the method comprises configuring the rows in each lane with perforations to separate the rows from each other.

In an additional aspect, a method of producing an individual absorbent article comprises removing at least one layer of absorbent articles from a container comprising a first stack of absorbent articles, a second stack of absorbent articles and a divider between the first stack and the second stack, in which each layer of the first stack and the second stack comprises a plurality of lanes of absorbent articles coupled to each other, and decoupling an absorbent article in the removed layer from other absorbent articles in the removed layer to provide the individual absorbent article.

In certain embodiments, the method comprises configuring the divider to move toward a remaining lane of the first stack after another lane is removed from the first stack. In other embodiments, the method comprises configuring the divider to comprise a U-shaped member, a Z-shaped member, a C-shaped member, a lattice member or an X-shaped member. In further examples, the method comprises configuring each layer with a plurality of lanes of the absorbent articles, in which perforations are present to separate the plurality of lanes from each other. In some examples, the method comprises configuring the rows in each lane with perforations to separate the rows from each other.

In an additional aspect, system for packaging absorbent articles comprising a feeding device configured to provide at least one layer of absorbent articles, and a support configured to receive a container and position the container to receive absorbent articles from the feeding device to provide at least a first stack of absorbent articles in the container and a second stack of absorbent articles, adjacent to the first stack, in the container, in which the container is configured to receive a divider between at least some portion of the first stack and the second stack, the divider sized and arranged to provide support to at least one lane of absorbent articles of the first stack is described.

In certain embodiments, the feeding device is configured to move back and forth to assist in stacking of the layers. In other embodiments, the support is configured to move back and forth to assist in stacking of the layers. In some examples, the feeding device is sized and arranged to feed parallel sheets of absorbent articles to permit simultaneous stacking of the first and second stacks in the container. In other examples, the feeding device is configured to move laterally to stack the second stack adjacent to the first stack. In further embodiments, the system comprises at least one perforation device configured to provide perforations between lanes of absorbent articles. In some instances, the system comprises at least one pressing device configured to provide a crease between layers of the stacks. In other instances, the system comprises at least one cutting device configured to terminate the first stack once stacked. In some examples, the system comprises a second feeding device configured to assist in stacking of the first stack and the second stack in the container. In further embodiments, the system comprises a feed roll configured to provide a backing layer to form the absorbent articles.

In another aspect, an absorbent article produced by the steps of removing at least one lane of absorbent articles from a container comprising (i) a first stack comprising a plurality of individual absorbent articles, in which the absorbent articles of the first stack are present in stacked layers with each layer of the first stack comprising a plurality of the individual, absorbent articles arranged in lanes, (ii) a second stack adjacent to the first stack, in which the second stack comprises a plurality of individual absorbent articles, in which the absorbent articles of the second stack are present in stacked layers with each layer of the second stack comprising a plurality of the individual, absorbent articles arranged in lanes, and (iii) a divider between at least some portion of the first stack and the second stack, the divider sized and arranged to provide support to at least one lane of absorbent articles of the first stack, and separating at least one absorbent article in the removed lane from the other absorbent articles in the removed lane is disclosed.

In an additional aspect, an absorbent article produced by the steps of removing at least one layer of absorbent articles from a container comprising (i) a first stack comprising a plurality of individual absorbent articles, in which the absorbent articles of the first stack are present in stacked layers with each layer of the first stack comprising a plurality of the individual, absorbent articles arranged in lanes, (ii) a second stack adjacent to the first stack, in which the second stack comprises a plurality of individual absorbent articles, in which the absorbent articles of the second stack are present in stacked layers with each layer of the second stack comprising a plurality of the individual, absorbent articles arranged in lanes, and (iii) a divider between at least some portion of the first stack and the second stack, the divider sized and arranged to provide support to at least one lane of absorbent articles of the first stack, and separating at least one absorbent article in the removed layer from the other absorbent articles in the removed layer is provided.

Additional configurations and attributes, features, aspects, and embodiments thereof are described in more detail herein.

### BRIEF DESCRIPTION OF THE FIGURES

Certain features, aspects and embodiments of the articles, systems and methods are described with reference to the accompanying figures, in which:

FIG. 1 is an illustration showing a top view of a stack of absorbent articles disposed in a container, in accordance with certain configurations;

FIGS. 2A and 2B show a top view of layered stacks comprising various perforations, in accordance with certain configurations;

FIGS. 3A and 3B show tops views of layered stacks comprising lanes of absorbent articles, in accordance with certain configurations;

FIGS. 4A and 4B show layered stacks of articles disposed in a container comprising a U-shaped divider, in accordance with certain configurations;

FIGS. 5A and 5B show a side view of a U-shaped divider, in accordance with certain configurations;

FIG. 6 is an illustration of a top view showing an inserted U-shaped divider comprising an inward projection, in accordance with certain examples;

FIGS. 7A and 7B show layered stacks of articles disposed in a container comprising a Z-shaped divider, in accordance with certain configurations;

FIGS. 8A and 8B show a side view of a Z-shaped divider, in accordance with certain configurations;

FIG. 9 is an illustration of a top view showing an inserted Z-shaped divider comprising an inward projection, in accordance with certain examples;

FIGS. 10A and 10B show layered stacks of articles disposed in a container comprising a C-shaped divider, in accordance with certain configurations;

FIGS. 11A and 11B show a side view of a C-shaped divider, in accordance with certain configurations;

FIG. 12 is an illustration of a top view showing an inserted C-shaped divider comprising an inward projection, in accordance with certain configurations;

FIG. 13 is an illustration showing an X-shaped divider, in accordance with certain configurations;

FIG. 14 is an illustration showing a lattice-shaped divider, in accordance with certain configurations;

FIGS. 15A and 15B show stacked layers of absorbent articles, in accordance with certain configurations;

FIG. 16 is an illustration of a system configured to stack absorbent articles into layers, in accordance with certain configurations;

FIGS. 17A and 17B are illustrations of container portions, in accordance with certain examples;

FIGS. 18A-18F are photographs showing a Z-shaped divider placed into a container comprising a first stack and a second stack;

FIGS. 19 and 20 are photographs showing a C-shaped divider inserted into a container; and

FIG. 21 is a photograph showing an expanded view of the Z-shaped divider.

It will be recognized by the person of ordinary skill in the art, given the benefit of this disclosure, that the sizes of the absorbent articles, dividers, and other components in the figures are not limiting and that additional components may also be included in any of the figures without departing from the spirit and scope of the technology described herein.

### DETAILED DESCRIPTION

Certain features, aspects and embodiments are described below with reference to absorbent articles. While not wishing to be limiting, the absorbent articles typically include one or more absorbent materials that can retain a liquid or liquids for at least some period. While illustrative uses of the absorbent materials are described below, additional suitable uses will be selected by the person of ordinary skill in the art, given the benefit of this disclosure.

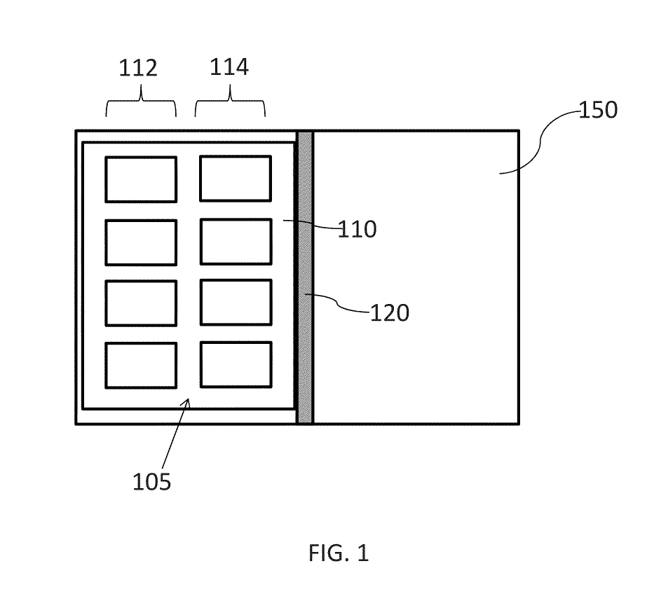

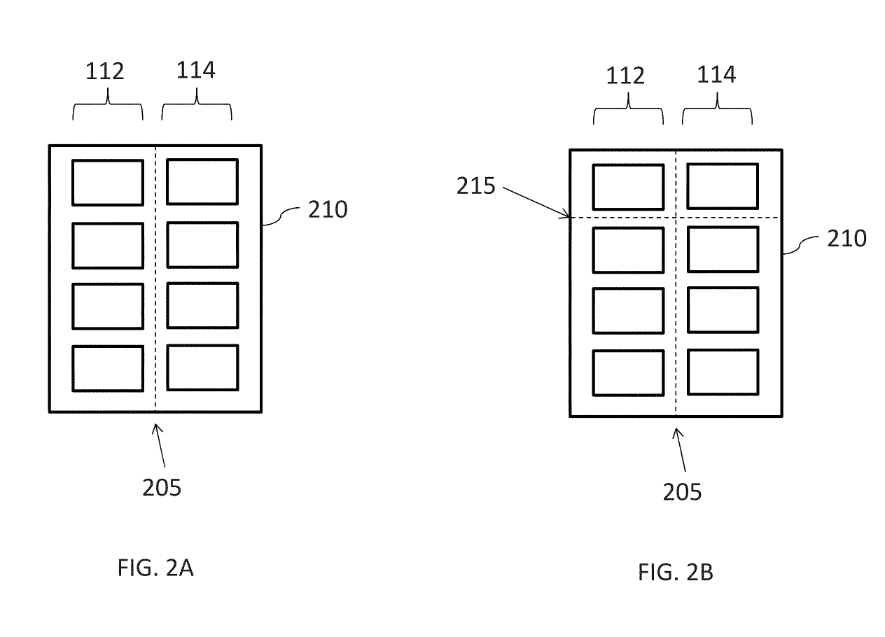

In certain embodiments, a top view of a stack 105 of absorbent articles placed in a container 150 is shown in FIG. 1. In each layer 110 of the stack 105, a plurality of absorbent articles are present. The absorbent articles can be arranged in lanes 112, 114. As shown in more detail in FIG. 2A, the lanes 112, 114 can be separated from each other through perforations 205 (or other structures such as slits, holes, etc.) in a support layer 210 to permit separation of the various lanes 112, 114. In addition, the rows of the lanes may also be separated by perforations 215 (or other structures such as slits, holes, etc.) in the support layer 210, as shown in FIG. 2B. While perforations 215 are shown between the upper and second rows in FIG. 2B, there may be perforations (or other suitable structures) between each row in each layer of the absorbent article stacks.

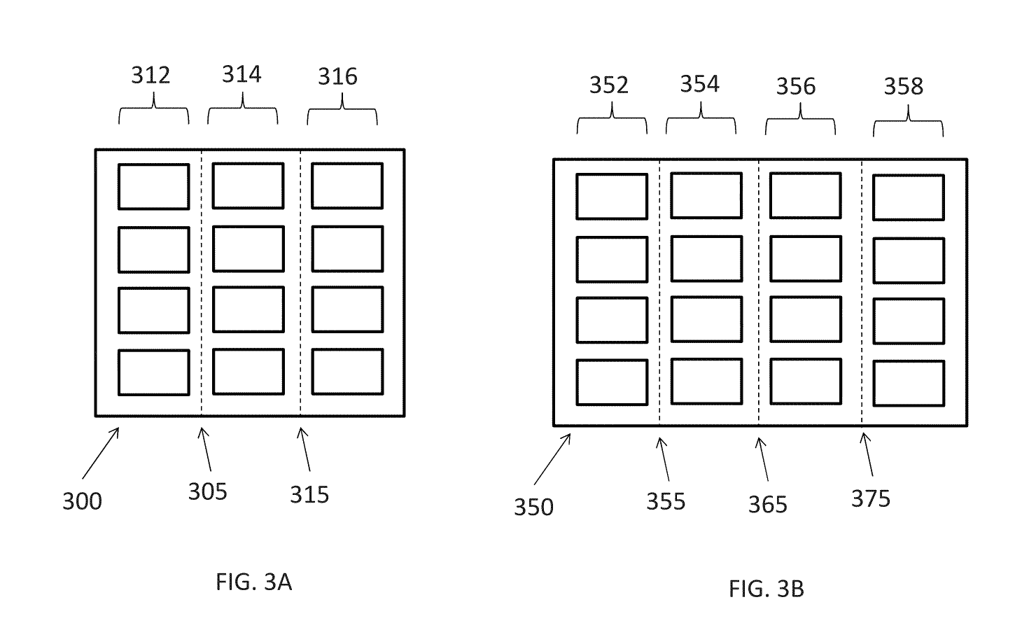

The exact number of lanes present in each layer may vary and may be the same or may be different in each of the layers of any one stack. In some instances, each of the layers may comprise two, three, four or more lanes in each layer. Referring to FIG. 3A, a layer comprising lanes 312, 314 and 316 of absorbent articles separated by perforations 305 and 315 is shown. Perforations may also be present between each of the rows present on the layer 300. Referring to FIG. 3B, a layer 350 comprising four lanes 352, 354, 356 and 358 is shown. Perforations 355, 365 and 375 separate the various lanes. If desired, perforations may also be present between the rows of the absorbent articles in the layer 350.

Referring again to FIG. 1, a divider 120 is shown as being inserted into the container 150. In certain configurations, the divider 120 can bisect the width of the container such that a first stack of absorbent articles, e.g., stack 105, is separated from a second stack of absorbent articles (not shown) by the divider 120. As noted in more detail herein, as the layers of the stacks in the containers are removed during a processing operation, an adjacent stack can become unstable and may fall over. In addition, placement of two stacks beside each other can result in interleaving of the layers of the stacks making it difficult to extract any one stack (or lane thereof) without interference from the other stack. In other uses, individual lanes of a particular stack can be removed leaving behind the remaining lanes in that stack. As fewer and fewer lanes remain in any one stack, the odds of the lanes collapsing or falling over increase. If the stacks or lanes were to fall, then the processing operation must be halted, and the fallen stacks (or lanes) are generally not suitable for use in an automated processing operation. By including a divider as described herein, interruptions in processing of the absorbent articles can be avoided, processing speed can be increased (if desired), and increased amounts of absorbent articles can be packaged into a single container.

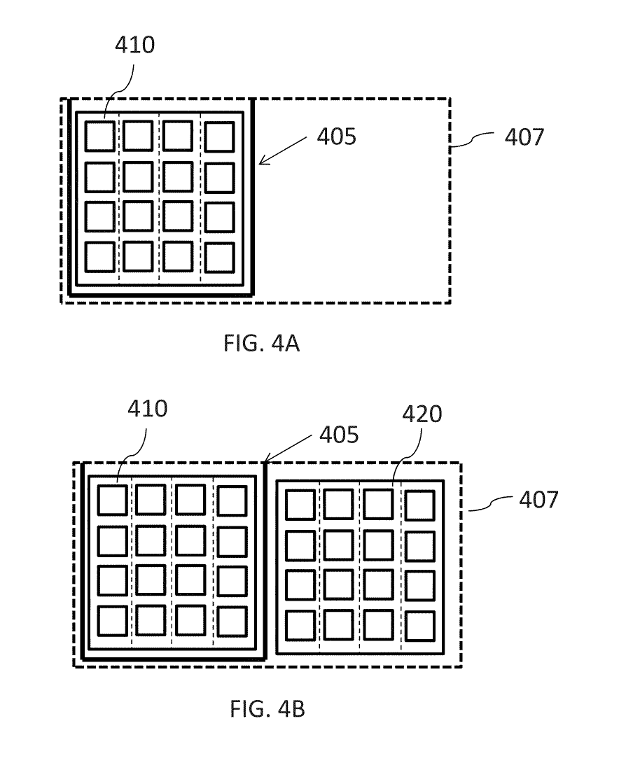

In certain examples, the exact configuration of the divider can vary. In some configurations, the divider is sized and arranged to be inserted into a single container that can hold at least two layered stacks of absorbent articles. Referring to FIG. 4A, one configuration of a divider is shown. The divider 405 is configured as a U-shaped member that has been inserted into a container 407. While the U-shaped divider is shown as having dimensions of the arms of about the width of the container, the width may be less than a width of the container if desired. A stack 410 of absorbent articles is shown for reference. While four lanes of absorbent articles are shown, fewer than four lanes or more than four lanes may be present. The arms of the U-shaped divider 405 generally run along the sides and bottom of the stack 410 to provide lateral support to the stack 410, though as noted herein the arm dimensions may be the same as that of the side of the stack, greater than the dimensions of the side of the stack or less than the dimensions of the side of the stack. The divider 405 may be integral to the container 407 so that it is not generally removable from the container 407 or may be inserted into the container 407 before, during or after the stack 410 is placed in the container 407. As lanes are removed from the stack 410, the divider 405 can provide lateral support to the remaining lanes to avoid collapse of those lanes. For example, as the left lane of the stack 410 is removed, leaving behind the three other lanes, the stack 410 may be less stable. This instability may increase further as the second and third lanes are removed leaving behind the fourth or terminal lane at the far right of the stack 410. While not wishing to be bound by any particular theory, as the overall size of the absorbent articles decrease, the instability of the terminal lane generally increases due to less support structure being present in the smaller articles. In some instances, the container may have a sloping bottom or can be tilted such that the stack 410 is biased toward the divider 405 under gravitational forces. This slope may be small, e.g., less than 5 degrees or 10 degrees to avoid angling the stack to a substantial degree that might interfere with automated removal of the absorbent articles in the stack 410.

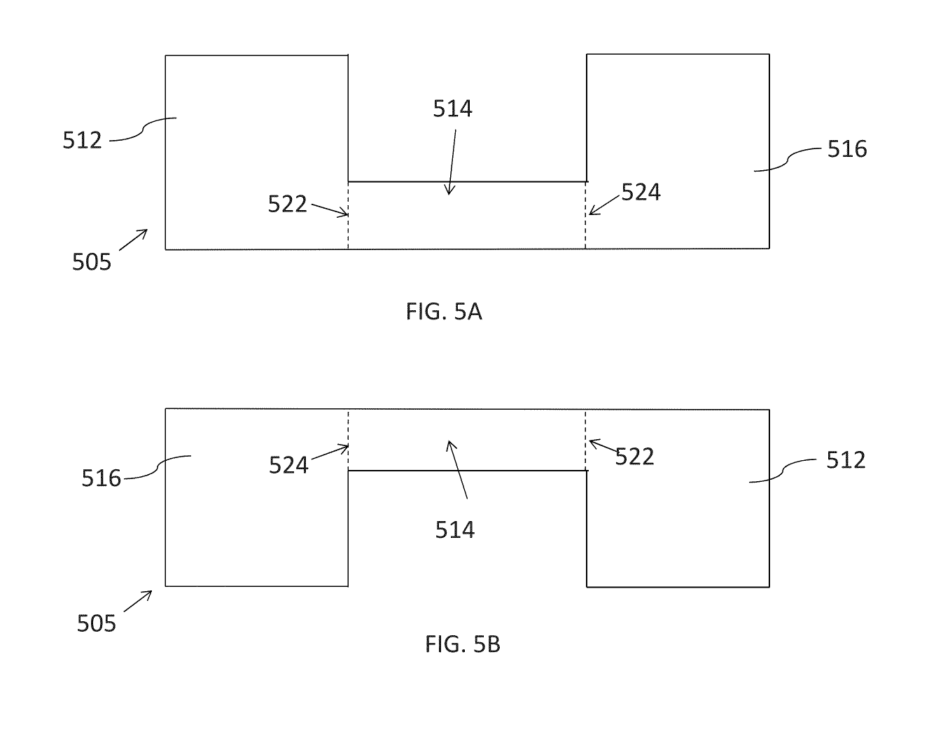

In certain configurations, the divider 405 may comprise one or more of a paper, a plastic, a metal, or combinations thereof. The divider may be a unitary structure with one or more bends or folds to provide the arms of the U-shaped divider 405 or may include hinges or other structures to provide the U-shape. In some instances, the divider 405 is configured as a laminate, e.g., may comprise a plastic backing and a paper cover layer or may comprise three or more different layers. In certain configurations, one or more portions or sections of the divider 405 may have an overall height less than a height of the stack 410. For example, and referring to FIG. 5A, a divider 505 is shown comprising three sections 512, 514 and 516. The height of the section 514 is generally less than the height of sections 512 and 516. In use, the divider 505 can be bent at axes 522, 524 to form the U-shaped member (when view from the top of the container). The section 514 can rest against the lower portion of the stack 510. If desired, the divider can be rotated 180 degrees and be used. Referring to FIG. 5B, the divider 505 is shown as being rotated 180 degrees such that after insertion into container, the portion 514 will rest against an upper side of the stack 410. The lower portion of the stack 410 will not be supported by the divider 505. While not shown, the height of the sections 512, 516 need not be the same. In addition, the height of any one of the sections 512, 514, 516 may vary across the width of the divider.

In certain examples, the U-shaped divider may comprise one or more coatings or materials disposed on an outer surface or may include such materials within the divider. For example, where the divider comprises a paper substrate, e.g., corrugated cardboard, a hydrophobic coating can be applied to prevent moisture absorption by the divider. In other instances, at least one of an antibacterial coating, an antifungal coating, an anti-static coating, and a friction reducing coating may be present on the U-shaped divider. In some instances, the divider may comprise an indicator, such as, for example, lot number, production date, height markings or other desired text, colors or markings.

Referring to FIG. 4B, the U-shaped divider is shown as being inserted between the stack 410 and a second stack 420. As described in more detail below, the stacks 410 and 420 are generally coupled to each other so that when all lanes (or layers) of the stack 410 have been removed, the last (bottom) layer of the stack 410 is coupled to the top layer of the stack 420. If desired, however, the stacks can be coupled at the bottom of both layered stacks or in other manners. By coupling the stacks to each other, automated removal of all lanes (or all layers) in both stacks 410, 420 can be performed without the need to stop removal of the absorbent articles when switching from the stack 410 to the stack 420. If desired, an additional divider (not shown) can be inserted into the container 407 to provide support to the second stack 420. Alternatively, the container 407 can include a sloped bottom or be tilted toward one side to provide a slight tilt of the stacks 410 and 420.

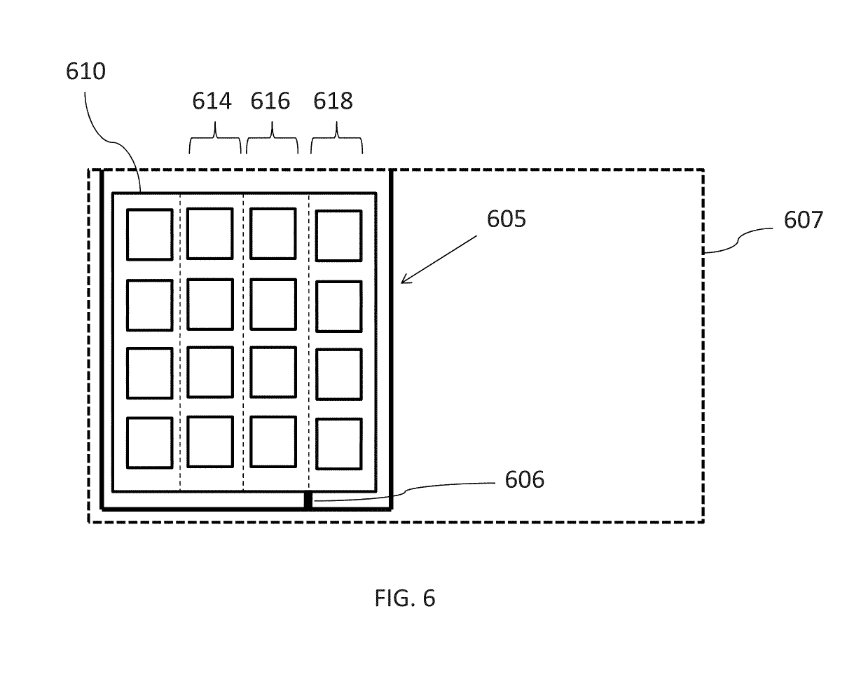

In certain configurations, the U-shaped divider may comprise one or more inward projections that rest against a surface of the stack. Referring to FIG. 6, a divider 605 is shown as a generally U-shaped member that comprises an inward projection 606. The divider 605 is placed in a container 607 comprising a stack 610 of layered absorbent articles. The projection 606 is constructed and arranged to engage the lower surface of the stack 610 adjacent to a perforation between lanes 616 and 618 of the stack 610. The terminal lane 618, after removal of the other lanes of the stack 610, may collapse due to insufficient supporting structure from material of the lane 618. The inward projection 606 can act to provide support to the side of the absorbent articles in lane 618 to deter collapse. The inward projection 606 is desirably sized large enough to provide support to the terminal lane 618 but not so large as to distort the other lanes during processing of the stack 610. In some instances, the inward projection may be about one inch to about six inches, for example. In other instances, the inward projection may rest on top of lane 616 and drop into position against lane 618 as material from lane 616 is removed. In this manner, no biasing or force is applied to lane 616 during the processing operation. While the projection 606 is shown at the bottom of the container 607 (in this top view), it instead may be placed at the top of the container 607 if the position of the divider 605 is altered so that the divider is placed around the left, top and right sides of the stack 610. In some instances, the divider 605 may comprise more than a single inward projection. For example, a second projection may be present that is positioned roughly between the lane 616 and lane 614 so that the projection can provide support to lane 616 when material from lane 614 is removed. The inward projection 606 may have a height that is roughly the same as the height of the stack 610 or may have a height that is less than that of the stack 610, e.g., may only support the bottom half, the top half or some other portion of the stack 610. While not shown, a second stack typically is present in the other side of the container 607 and may be coupled to the stack 610 through at least one layer, e.g., a top layer of the second stack can be coupled to a bottom layer of the stack 610.

In some embodiments, the U-shaped divider may comprise a moveable component that can be placed against the side of the stack. The moveable component may be biased toward the side of the stack using a spring, piston or the like such that removal of one lane of the stack permits the moveable component to engage an adjacent lane of the stack. This movement may continue until all lanes of the stack are removed. In some instances, the left arm of the U-shaped member can be coupled to the bottom portion of the U-shaped member through a spring-loaded slot. As lanes are removed from the stack, the moveable component slides along the slot until it reaches the right arm of the U-shaped member. The spring force may be effective to provide support but not so high as to distort the lanes in the stack. Other mechanisms and devices to bias one of the U-shaped arms against some portion of the side of a stack will be recognized by the person of ordinary skill in the art, given the benefit of this disclosure.

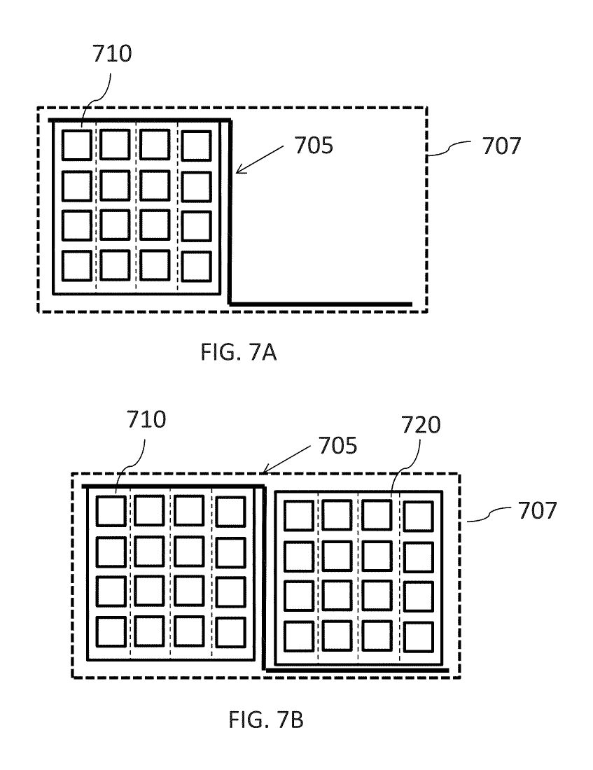

In other configurations, the divider may be configured as a Z-shaped member. Referring to FIG. 7A, a top view of one configuration of a Z-shaped divider is shown. The divider 705 runs along a top side of the stack 710, along the right side of the stack 710 (comprising a plurality of absorbent articles) and along a bottom surface of a second stack 720 (as shown in FIG. 7B). While the Z-shaped divider is shown as having certain dimensions of about one-half the length of the container 707, the various sections of the Z-shaped divider can be smaller or larger than what is shown in FIG. 7. If desired, the divider may instead be configured as a backward “Z” with the divider running along the bottom surface of the stack 710, along the right side of the stack 710 and a top side of the stack 720. While four lanes of absorbent articles are shown in the stacks of FIGS. 7A and 7B, fewer than four lanes or more than four lanes may be present. The divider 705 may be integral to the container 707 so that it is not generally removable from the container 707 or may be inserted into the container 707 before, during or after the stack 710 is being placed in the container 707. As lanes are removed from the stack 710, the divider 705 can provide lateral support to the remaining lanes to avoid collapse of those lanes. For example, as the left lane of the stack 710 is removed, leaving behind the three other lanes, the stack 710 may be less stable. This instability may increase further as the second and third lanes are removed leaving behind the fourth or terminal lane at the far right of the stack 710. While not wishing to be bound by any particular theory, as the overall size of the absorbent articles decrease, the instability of the terminal lane of the stack 710 generally increases due to less support structure being present in the smaller articles. In some instances, the container 707 may have a sloping bottom or can be tilted such that the stack 710 is biased toward the divider 705 under gravitational forces. This slope may be small, e.g., less than 5 degrees or 10 degrees to avoid angling the stack to a substantial degree that might interfere with automated removal of the absorbent articles in the stack 710.

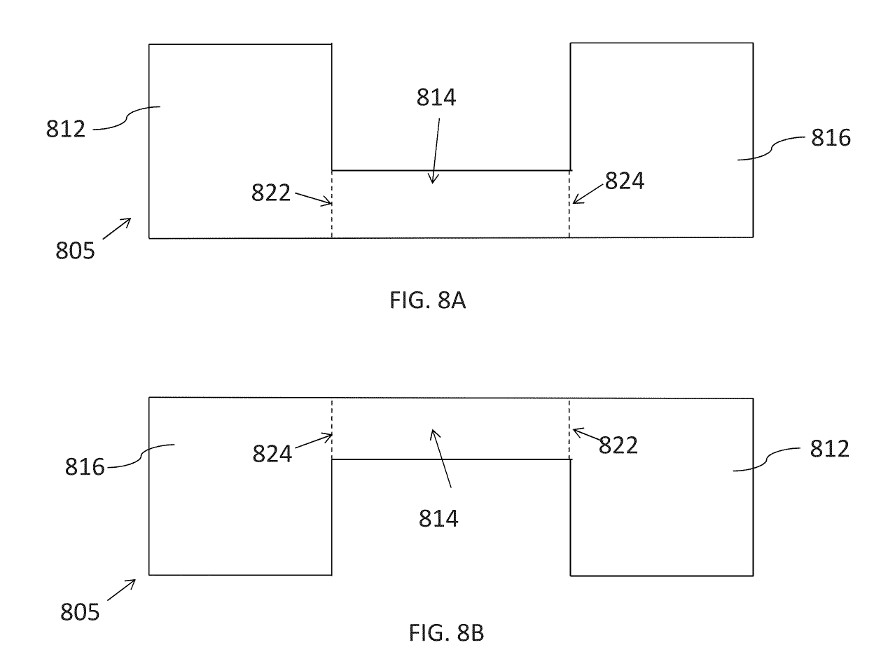

In certain configurations, the Z-shaped divider 705 may comprise one or more a paper, a plastic, a metal, or combinations thereof. The divider may be a unitary structure with one or more bends or folds to permit the divider to be bent into a Z-shape or may include hinges or other structures to provide the Z-shape. In some instances, the divider 705 is configured as a laminate, e.g., may comprise a plastic backing and a paper cover layer or may comprise three or more different layers. In certain configurations, one or more portions or sections of a divider may have an overall height less than a height of the stack 710. For example, and referring to FIG. 8A, a divider 805 is shown comprising three sections 812, 814 and 816. The height of the section 814 is generally less than the height of sections 812 and 816. In use, the divider 805 can be bent at axes 822, 824 to form the Z-shaped member (when view from the top of the container). The section 814 can rest against the lower portion of the stack. If desired, the divider can be rotated 180 degrees and be used. Referring to FIG. 8B, the divider 805 is shown as being rotated 180 degrees such that after insertion into container, the portion 814 will rest against an upper side of the stack. The lower portion of the stack 810 will not be supported by the divider 805. While not shown, the height of the sections 812, 816 need not be the same. In addition, the height of any one of the sections 812, 814, 816 may vary across the width of the divider when in an extended form.

In certain examples, the Z-shaped divider may comprise one or more coatings or materials disposed on an outer surface or may include such materials within the divider. For example, where the divider comprises a paper substrate, e.g., corrugated cardboard, a hydrophobic coating can be applied to prevent moisture absorption by the divider. In other instances, at least one of an antibacterial coating, an antifungal coating, an anti-static coating, and a friction reducing coating may be present on the Z-shaped divider. In some instances, the divider may comprise an indicator, such as, for example, lot number, production date, height markings or other desired text, colors or markings.

Referring to FIG. 7B, the Z-shaped divider is shown as being inserted between the stack 710 and a second stack 720. As described in more detail below, the stacks 710 and 720 are generally coupled to each other so that when all lanes (or layers) of the stack 710 have been removed, the last (bottom) layer of the stack 710 is coupled to the top layer of the stack 720. If desired, however, the stacks can be coupled at the bottom of both layered stacks or in other manners. By coupling the stacks to each other, automated removal of all lanes (or all layers) in both stacks 710, 720 can be performed without the need to stop removal of the absorbent articles when switching from the stack 710 to the stack 720. If desired, an additional divider (not shown) can be inserted into the container 707 to provide support to the second stack 720. Alternatively, the container 707 can include a sloped bottom or be tilted toward one side to provide a slight tilt of the stacks 710 and 720.

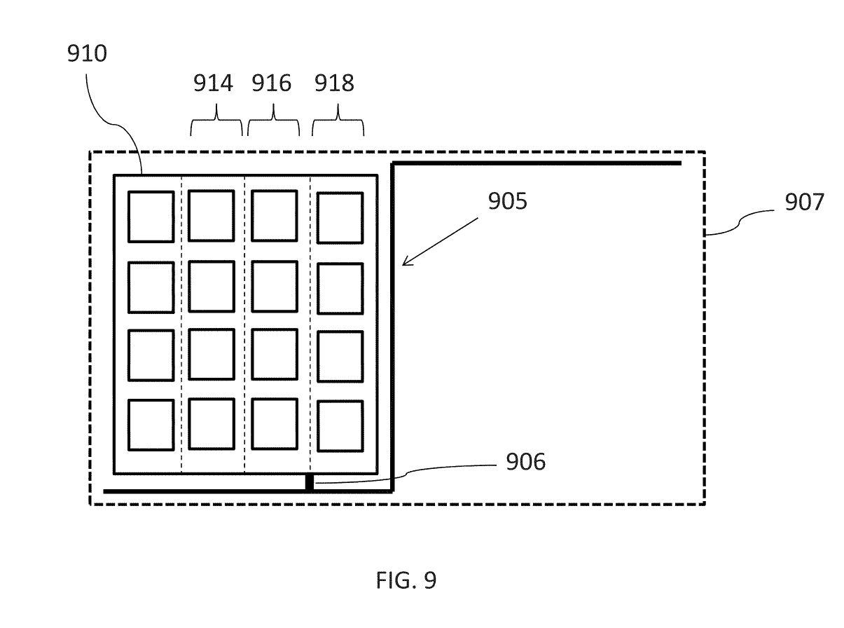

In certain configurations, the Z-shaped divider may comprise one or more inward projections that rest against a surface of the stack. Referring to FIG. 9, a divider 905 is shown as a generally Z-shaped member that comprises an inward projection 906. The divider 905 is placed in a container 907 comprising a stack 910 of layered absorbent articles. The projection 906 is constructed and arranged to engage the lower surface of the stack 910 adjacent to a perforation between lanes 916 and 918 of the stack 910. The terminal lane 918, after removal of the other lanes of the stack 910, may collapse due to insufficient supporting structure from material of the lane 918. The inward projection 906 can act to provide support to the side of the absorbent articles in lane 918 to deter collapse. The inward projection 906 is desirably sized large enough to provide support to the terminal lane 918 but not so large as to distort the other lanes during processing of the stack 910. In some instances, the inward projection may be about one inch to about six inches, for example. In other instances, the inward projection may rest on top of lane 916 and drop into position against lane 918 as material from lane 916 is removed. In this manner, no biasing or force is applied to lane 916 during the processing operation. While the projection 906 is shown at the bottom of the container 907 (in this top view), it instead may be placed at the top of the container 907 if the position of the divider 905 is altered so that the divider is placed around the left, top and right sides of the stack 910. In some instances, the divider 905 may comprise more than a single inward projection. For example, a second projection may be present that is positioned roughly between lane the lane 916 and lane 914 so that the projection can provide support to lane 916 when material from lane 914 is removed. The inward projection 906 may have a height that is roughly the same as the height of the stack 910 or may have a height that is less than that of the stack 910, e.g., may only support the bottom half, the top half or some other portion of the stack 910. While not shown, a second stack typically is present in the other side of the container 907 and may be coupled to the stack 910 through at least one layer, e.g., a top layer of the second stack can be coupled to a bottom layer of the stack 910.

In some embodiments, the Z-shaped divider may comprise a moveable component that can be placed against the side of the stack. The moveable component may be biased toward the side of the stack using a spring, piston or the like such that removal of one lane of the stack permits the moveable component to engage an adjacent lane of the stack. This movement may continue until all lanes of the stack are removed. In some instances, the Z-shaped member may further include an additional arm on the left-side of the Z-shaped member to couple to the bottom portion of the Z-shaped member through a spring-loaded slot. As lanes are removed from the stack, the moveable component slides along the slot until it reaches the central portion of the Z-shaped member. The spring force may be effective to provide support but not so high as to distort the lanes in the stack. Other mechanisms and devices to provide a biasing force using the Z-shaped divider will be recognized by the person of ordinary skill in the art, given the benefit of this disclosure. In some instances, the inward projection may be moveable along some portion of the Z-shaped divider to provide a biasing force against the side of the stack.

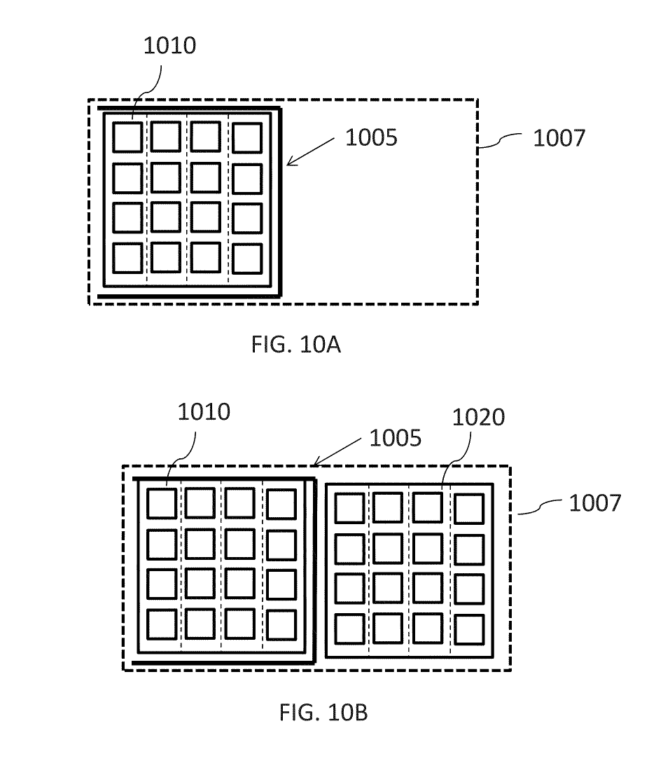

In certain instances, the divider may be configured as a C-shaped member. Referring to FIG. 10A, a top view of one configuration of a C-shaped divider is shown. The divider 1005 runs along a top side of the stack 1010, along the right side of the stack 1010 (comprising a plurality of absorbent articles) and along a bottom surface of the stack 1010. If desired, any one dimension, e.g., width, length or both, of a section of the C-shaped divider can be less than that of the stack 1010 or can be greater than that of the stack 1010. If desired, the divider may instead be configured with the divider running along the bottom surface of the stack 1010, along the left side of the stack 1010 and a top side of the stack 1020. While 4 lanes of absorbent articles are shown in the stacks of FIGS. 10A and 10B, fewer than four lanes or more than four lanes may be present. The divider 1005 may be integral to the container 1007 so that it is not generally removable from the container 1007 or may be inserted into the container 1007 before, during or after the stack 1010 is being placed in the container 1007. As lanes are removed from the stack 1010, the divider 1005 can provide lateral support to the remaining lanes to avoid collapse of those lanes. For example, as the left lane of the stack 1010 is removed, leaving behind the three other lanes, the stack 1010 may be less stable and tend to move or shift. This instability may increase further as the second and third lanes are removed leaving behind the fourth or terminal lane at the far right of the stack 1010. While not wishing to be bound by any particular theory, as the overall size of the absorbent articles decrease, the instability of the terminal lane of the stack 1010 generally increases due to less support structure being present in the smaller articles. In some instances, the container 1007 may have a sloping bottom or can be tilted such that the stack 1010 is biased toward the divider 1005 under gravitational forces. This slope may be small, e.g., less than 5 degrees or 10 degrees to avoid angling the stack to a substantial degree that might interfere with automated removal of the absorbent articles in the stack 1010.

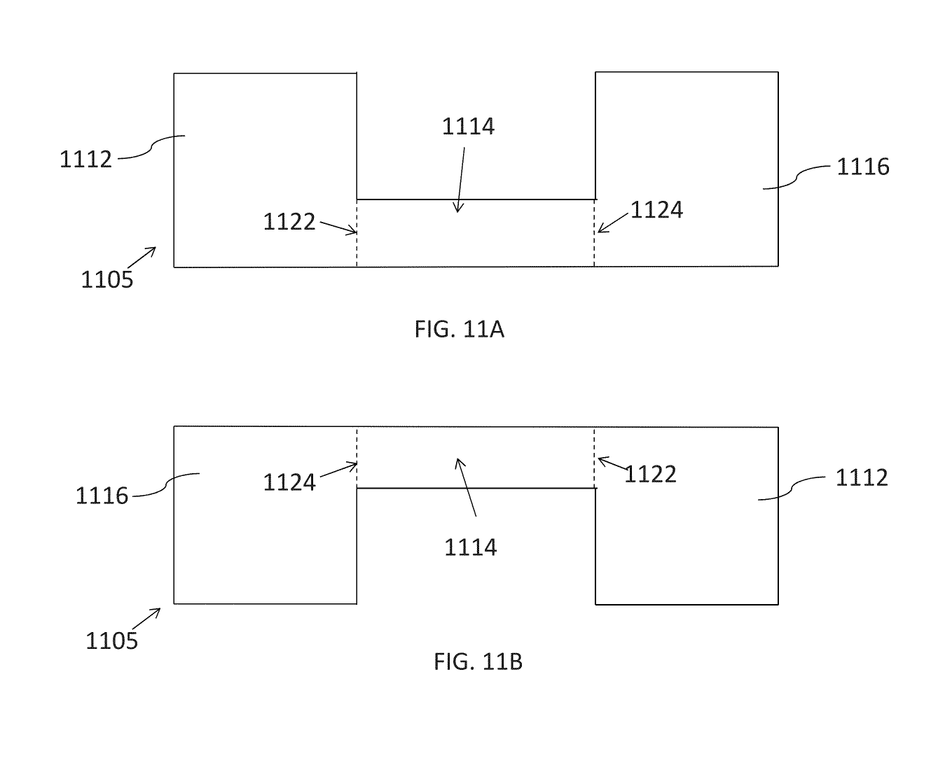

In certain configurations, the C-shaped divider 1005 may comprise one or more of a paper, a plastic, a metal, or combinations thereof. The C-shaped divider may be a unitary structure with one or more bends or folds to permit the divider to be bent into a C-shape or may include hinges or other structures to provide the C-shape. In some instances, the divider 1005 is configured as a laminate, e.g., may comprise a plastic backing and a paper cover layer or may comprise three or more different layers. In certain configurations, one or more portions or sections of a divider may have an overall height less than a height of the stack 1010. For example, and referring to FIG. 11A, a divider 1105 is shown comprising three sections 1112, 1114 and 1116. The height of the section 1114 is generally less than the height of sections 1112 and 1116. In use, the divider 1105 can be bent at axes 1122, 1124 to form the Z-shaped member (when view from the top of the container). The section 1114 can rest against the lower portion of the stack. If desired, the divider can be rotated 180 degrees and be used. Referring to FIG. 11B, the divider 1105 is shown as being rotated 180 degrees such that after insertion into container, the portion 1114 will rest against an upper side of the stack. The lower portion of the stack 1110 will not be supported by the divider 1105. While not shown, the height of the sections 1112, 1116 need not be the same. In addition, the height of any one of the sections 1112, 1114, 1116 may vary across the width of the divider when in an extended form.

In certain examples, the C-shaped divider may comprise one or more coatings or materials disposed on an outer surface or may include such materials within the divider. For example, where the divider comprises a paper substrate, e.g., corrugated cardboard, a hydrophobic coating can be applied to prevent moisture absorption by the divider. In other instances, at least one of an antibacterial coating, an antifungal coating, an anti-static coating, and a friction reducing coating may be present on the C-shaped divider. In some instances, the divider may comprise an indicator, such as, for example, lot number, production date, height markings or other desired text, colors or markings.

Referring to FIG. 10B, the C-shaped divider is shown as being inserted between the stack 1010 and a second stack 1020. As described in more detail below, the stacks 1010 and 1020 are generally coupled to each other so that when all lanes (or layers) of the stack 1010 have been removed, the last (bottom) layer of the stack 1010 is coupled to the top layer of the stack 1020. If desired, however, the stacks can be coupled at the bottom of both layered stacks or in other manners. By coupling the stacks to each other, automated removal of all lanes (or all layers) in both stacks 1010, 1020 can be performed without the need to stop removal of the absorbent articles when switching from the stack 1010 to the stack 1020. If desired, an additional divider (not shown) can be inserted into the container 1007 to provide support to the second stack 1020. Alternatively, the container 1007 can include a sloped bottom or be tilted toward one side to provide a slight tilt of the stacks 1010 and 1020.

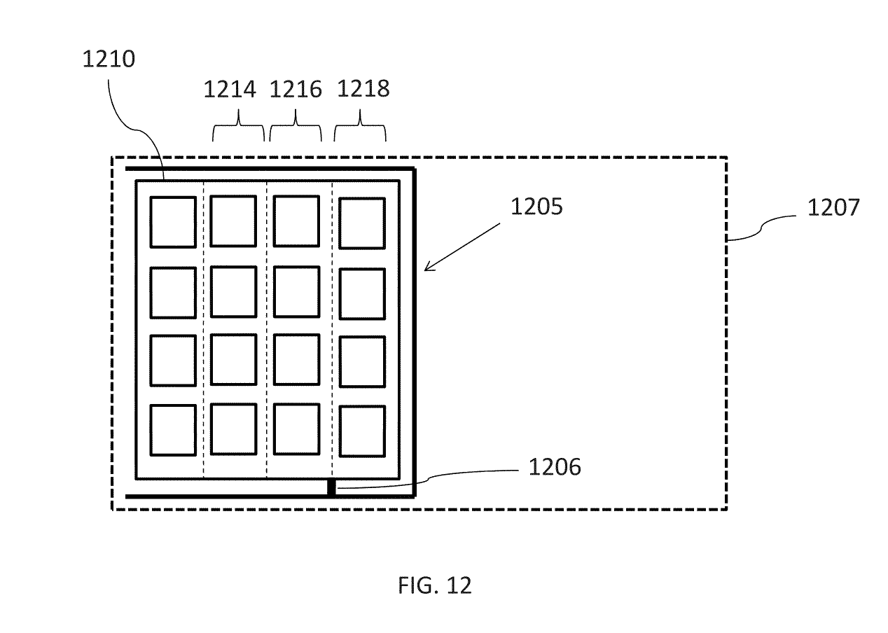

In certain configurations, the C-shaped divider may comprise one or more inward projections that rest against a surface of the stack. Referring to FIG. 12, a divider 1205 is shown as a generally backward C-shaped member that comprises an inward projection 1206. The divider 1205 is placed in a container 1207 comprising a stack 1210 of layered absorbent articles. The projection 1206 is constructed and arranged to engage the lower surface of the stack 1210 adjacent to a perforation between lanes 1216 and 1218 of the stack 1210. The terminal lane 1218, after removal of the other lanes of the stack 1210, may collapse due to insufficient supporting structure from material of the lane 1218. The inward projection 1206 can act to provide support to the side of the absorbent articles in lane 1218 to deter collapse. The inward projection 1206 is desirably sized large enough to provide support to the terminal lane 1218 but not so large as to distort the other lanes during processing of the stack 1210. In some instances, the inward projection may be about one inch to about six inches, for example. In other instances, the inward projection may rest on top of lane 1216 and drop into position against lane 1218 as material from lane 1216 is removed. In this manner, no biasing or force is applied to lane 1216 during the processing operation. While the projection 1206 is shown at the bottom of the container 1207 (in this top view), it instead may be placed at the top of the container 1207. In some instances, the divider 1205 may comprise more than a single inward projection. For example, a second projection may be present that is positioned roughly between lane the lane 1216 and lane 1214 so that the projection can provide support to lane 1216 when material from lane 1214 is removed. The inward projection 1206 may have a height that is roughly the same as the height of the stack 1210 or may have a height that is less than (or more than) that of the stack 1210, e.g., may only support the bottom half, the top half or some other portion of the stack 1210. While not shown, a second stack typically is present in the other side of the container 1207 and may be coupled to the stack 1210 through at least one layer, e.g., a top layer of the second stack can be coupled to a bottom layer of the stack 1210.

In some embodiments, the C-shaped divider may comprise a moveable component that can be placed against the side of the stack. The moveable component may be biased toward the side of the stack using a spring, piston or the like such that removal of one lane of the stack permits the moveable component to engage an adjacent lane of the stack. This movement may continue until all lanes of the stack are removed. In some instances, the C-shaped member may further include an additional arm on the left-side of the C-shaped member to couple the bottom portion of the C-shaped member through a spring-loaded slot. As lanes are removed from the stack, the moveable component slides along the slot until it reaches the central portion of the C-shaped member. The spring force may be effective to provide support but not so high as to distort the lanes in the stack. Other mechanisms and devices to provide a biasing force using the C-shaped divider will be recognized by the person of ordinary skill in the art, given the benefit of this disclosure. In some instances, the inward projection may be moveable along some portion of the C-shaped divider to provide a biasing force against the side of the stack. In certain instances, the C-shaped member can be configured with a movable component such that a regular “C shape” is present when all lanes of the stack are present, and a backward “C shape” results from movement of the movable component along the upper and lower components of the C-shaped member.

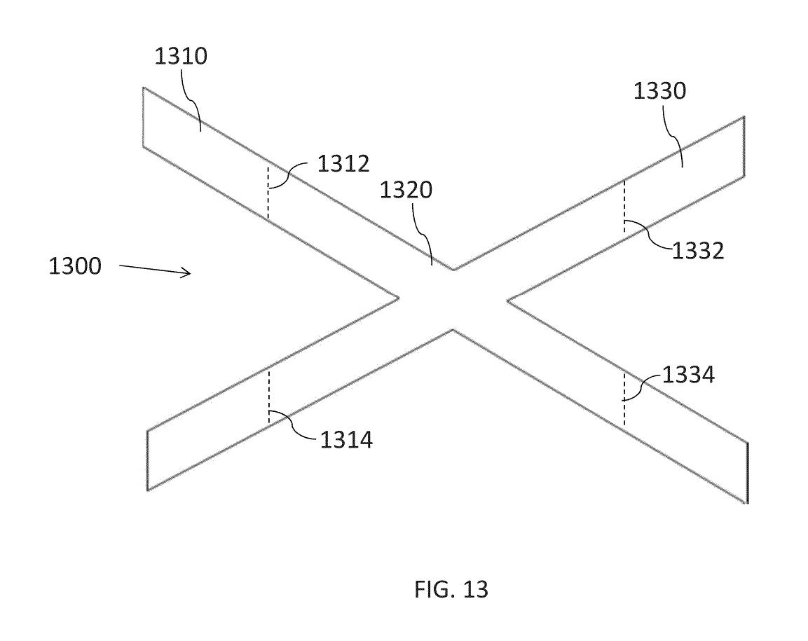

In certain instances, the divider may be configured as an X-shaped member. The X-shaped member may be inserted into a container and may resemble a Z-shape, a C-shape a U-shape (or other shapes) when viewed from the top of the container. Referring to FIG. 13, a side view of an X-shaped divider is shown. The X-shaped divider 1300 comprises two arms and may be divided into sections 1310, 1320 and 1330, if desired, though sections are not required. The sections may be bent about axes 1312, 1314, 1332 and 1334 to provide a desired shape (when viewed from the top), e.g., a Z-shape, U-shape, C-shape or other shapes. In certain configurations, the X-shaped divider 1300 may comprise one or more a paper, a plastic, a metal, or combinations thereof. The X-shaped divider may be a unitary structure with one or more bends or folds to permit the divider to be bent into a desired shape or may include hinges or other structures. In some instances, the divider 1300 is configured as a laminate, e.g., may comprise a plastic backing and a paper cover layer or may comprise three or more different layers. In certain configurations, one or more portions or sections of the divider 1300 may have an overall height less than a height of a stack. In certain examples, the X-shaped divider may comprise one or more coatings or materials disposed on an outer surface or may include such materials within the divider. For example, where the divider comprises a paper substrate, e.g., corrugated cardboard, a hydrophobic coating can be applied to prevent moisture absorption by the divider. In other instances, at least one of an antibacterial coating, an antifungal coating, an anti-static coating, and a friction reducing coating may be present on the X-shaped divider. In some instances, the divider may comprise an indicator, such as, for example, lot number, production date, height markings or other desired text, colors or markings. If desired, the X-shaped divider may comprise one or more inward projections that rest against a surface of the stack. The projection can be constructed and arranged to engage the lower surface of the stack adjacent to a perforation between lanes of the stack. The inward projection can act to provide support to the side of the absorbent articles in lane to deter collapse. The inward projection is desirably sized large enough to provide support to the terminal lane but not so large as to distort the other lanes during processing of the stack. In some instances, the inward projection may be about one inch to about six inches, for example. In other instances, the inward projection may rest on top of lane and drop into position against lane as material from lane is removed. In this manner, no biasing or force is applied to the lanes during the processing operation. In some instances, the divider 1300 may comprise more than a single inward projection. The inward projection may have a height that is roughly the same as the height of the stack or may have a height that is less than (or more than) that of the stack, e.g., may only support the bottom half, the top half or some other portion of the stack.

In some embodiments, the X-shaped divider may comprise a moveable component that can be placed against the side of the stack. The moveable component may be biased toward the side of the stack using a spring, piston or the like such that removal of one lane of the stack permits the moveable component to engage an adjacent lane of the stack. This movement may continue until all lanes of the stack are removed. In some instances, the X-shaped member may further include an additional arm or component to couple the bottom portion of the X-shaped member through a spring-loaded slot. As lanes are removed from the stack, the moveable component slides along the slot until it reaches the central portion of the X-shaped member. The spring force may be effective to provide support but not so high as to distort the lanes in the stack. Other mechanisms and devices to provide a biasing force using the X-shaped divider will be recognized by the person of ordinary skill in the art, given the benefit of this disclosure. In some instances, the inward projection may be moveable along some portion of the X-shaped divider to provide a biasing force against the side of the stack.

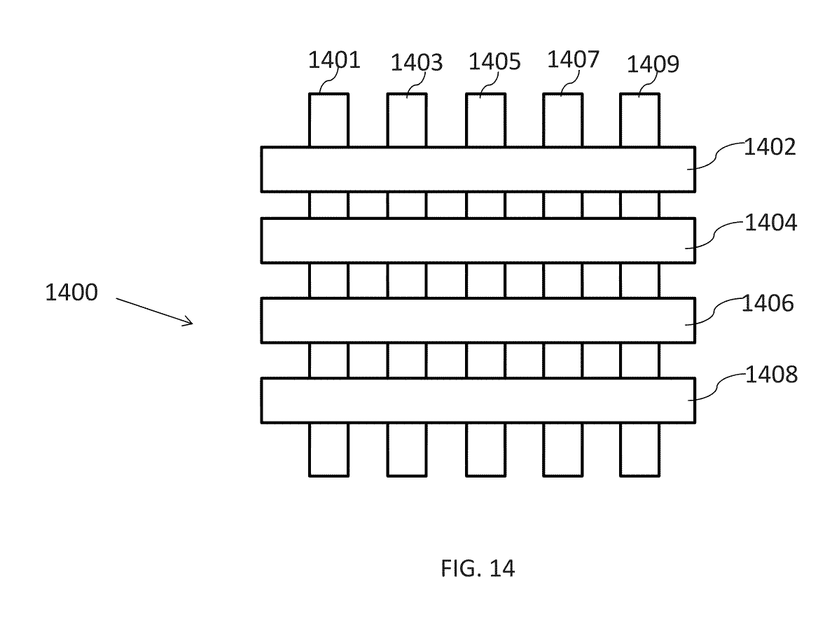

In certain configurations, the divider may be configured as a lattice-shaped member. The lattice-shaped member may be inserted into a container and may resemble a Z-shape, a C-shape a U-shape (or other shapes) when viewed from the top of the container. Referring to FIG. 14, a side view of a lattice shaped divider is shown. The lattice-shaped divider 1400 comprises a plurality of vertical sections 1401, 1403, 1405, 1407 and 1409 and a plurality of horizontal sections 1402, 1404, 1406 and 1408. The exact number of horizontal and vertical sections may vary and may be the same in number or may be different in number. Similarly, the dimensions of the horizontal sections may be the same or different than the vertical sections, and the dimensions of each horizontal section or vertical section need not be the same as the dimensions of other horizontal sections or vertical sections. The sections may be bent about axes to provide a desired shape (when viewed from the top), e.g., a Z-shape, U-shape, C-shape or other shapes. In certain configurations, the lattice-shaped divider may comprise one or more a paper, a plastic, a metal, or combinations thereof. The lattice-shaped divider may be a unitary structure with one or more bends or folds to permit the divider to be bent into a desired shape or may include hinges or other structures. In some instances, the divider 1400 is configured as a laminate, e.g., may comprise a plastic backing and a paper cover layer or may comprise three or more different layers. In certain configurations, one or more portions or sections of the divider 1400 may have an overall height less than a height of a stack. In certain examples, the lattice-shaped divider may comprise one or more coatings or materials disposed on an outer surface or may include such materials within the divider. For example, where the divider 1400 comprises a paper substrate, e.g., corrugated cardboard, a hydrophobic coating can be applied to prevent moisture absorption by the divider. In other instances, at least one of an antibacterial coating, an antifungal coating, an anti-static coating, and a friction reducing coating may be present on the lattice-shaped divider. In some instances, the divider 1400 may comprise an indicator, such as, for example, lot number, production date, height markings or other desired text, colors or markings. If desired, the lattice-shaped divider may comprise one or more inward projections that rest against a surface of the stack. The projection can be constructed and arranged to engage the lower surface of the stack adjacent to a perforation between lanes of the stack. The inward projection can act to provide support to the side of the absorbent articles in lane to deter collapse. The inward projection is desirably sized large enough to provide support to the terminal lane but not so large as to distort the other lanes during processing of the stack. In some instances, the inward projection may be about one inch to about six inches, for example. In other instances, the inward projection may rest on top of lane and drop into position against lane as material from lane is removed. In this manner, no biasing or force is applied to the lanes during the processing operation. In some instances, the divider 1400 may comprise more than a single inward projection. The inward projection may have a height that is roughly the same as the height of the stack or may have a height that is less than (or more than) that of the stack, e.g., may only support the bottom half, the top half or some other portion of the stack.

In some embodiments, the lattice-shaped divider may comprise a moveable component that can be placed against the side of the stack. The moveable component may be biased toward the side of the stack using a spring, piston or the like such that removal of one lane of the stack permits the moveable component to engage an adjacent lane of the stack. This movement may continue until all lanes of the stack are removed. In some instances, the lattice-shaped member may further include an additional arm or component to couple the bottom portion of the lattice-shaped member through a spring-loaded slot. As lanes are removed from the stack, the moveable component slides along the slot until it reaches the central portion of the lattice-shaped member. The spring force may be effective to provide support but not so high as to distort the lanes in the stack. Other mechanisms and devices to provide a biasing force using the lattice-shaped divider will be recognized by the person of ordinary skill in the art, given the benefit of this disclosure. In some instances, the inward projection may be moveable along some portion of the lattice-shaped divider to provide a biasing force against the side of the stack.

In some examples each absorbent article present in a layer may comprise a core layer and a backing layer. The term layer, as used in reference to each absorbent article, is used herein for convenience purposes only and the various materials used in the absorbent articles can be configured as a film, sheet, patches or take other forms. In use of the absorbent articles, the core layer typically contacts an object (not shown) and is effective to absorb fluid from the object. The backing layer typically provides a supportive structure to the core layer. In some embodiments, the backing layer may have a total weight of at least 50 grams per square meter (gsm), more particularly at least 90 gsm, at least 100 gsm or at least 150 gsm. In certain embodiments, the backing layer can have a desirable lamination strength. In certain embodiments, the core material can comprise a material effective to absorb and/or retain fluids. In some examples, the core material 110 can comprise a polar material. In other examples, the core material can comprise a material comprising a plurality of hydroxyl groups. In yet other examples, the core material can comprise cellulose or cellulose-based materials. In some embodiments, the core material can comprise a fiber mat comprising cellulose fibers sized and arranged as a loose non-woven material. In some embodiments, the core material can comprise a basis weight of about 100 grams/square meter to about 175 grams/square meter, e.g., about 110 g/square meter to about 150 grams/square meter. In some instances, different core materials may be present in different absorbent articles in a particular layer.

In certain examples, the core layer can be selected such that fluid that contacts the article becomes absorbed and trapped by the core layer. In certain instances, the physical and mechanical properties of the core layer can be selected to provide desired properties to the article. In some configurations, the basis weight of the core can be from about 80 gsm to about 160 gsm, more particularly about 100 gsm to about 150 gsm, for example, about 110 gsm to about 140 gsm. In certain embodiments, the basis weight of the core can be from one or more different materials present in the core. For example, where an absorbent material such as a superabsorber is present, the absorbent material may be present from around 5 gsm to about 50 gsm, more particularly about 10 gsm to about 40 gsm, for example about 25-35 gsm or 30 gsm. In some embodiments, the core may also include tissue, pulp or other types of paper or cellulose based material that can be present, for example, from about 20 gsm to about 100 gsm. Where tissue is present, the tissue can be present, for example, at a basis weight of about 15 gsm to about 60 gsm, more particularly about 25 gsm to about 50 gsm, e.g., about 30 gsm to about 45 gsm. Where pulp is present in the core, the pulp can be present from about 30 gsm to about 80 gsm or from about 40 gsm to about 70 gsm, for example about 45 gsm to about 65 gsm. The particular thickness of the core material may also vary depending on the desired use of the article. In some embodiments, the core material can be about 0.4 mm to about 2 mm thick, more particularly about 0.7 mm to about 1.5 mm thick, for example, about 0.8 mm thick to about 1 mm thick.

In certain embodiments, the pattern (if present) on the core material or core layers may vary depending on the desired aesthetic appearance and/or equipment used to produce the article. In some embodiments, one or more of a pinpoint pattern, square pattern, circular pattern, triangular pattern, rectangular pattern, hexagonal pattern or other geometric shapes may be present. In some instances as described herein, a pattern may be imparted to the core layer by using a roller comprising the particular pattern during manufacture of the article. In certain examples, the thickness of the overall article can vary from about 1 mm to about 2 mm, more particularly by about 1.2 mm to about 1.7 mm. In addition, the thickness of the article need not be uniform.

In other embodiments, additional materials can be present in the core layer, backing layer or both. For example, in certain embodiments one or more superabsorbent materials can be added, mixed with or otherwise disposed in the core material either prior to processing, during processing or after processing. Without wishing to be bound by any particular scientific theory, a superabsorber can act to absorb and/or retain large amounts of fluid. Illustrative superabsorbers are described, for example, in WO94/10596 and specific superabsorbers include, for example, acrylate composites, sodium polyacrylate, an acrylonitrile polymer, a polyacrylamide copolymer, an ethylene maleic anhydride copolymer, a cross-linked carboxymethylcellulose, a polyvinyl alcohol copolymer, a cross-linked polyethylene oxide, a starch grafted copolymer of polyacrylonitrile and other commonly available superabsorbent polymeric materials. In some embodiments, the process used to provide the core material may not include the use of any binders. In certain embodiments, the process used to provide the core material may be performed at room temperature and/or in ambient air. In certain examples, the core material can be produced as described in U.S. Pat. No. 6,675,702, the entire disclosure of which is incorporated herein by reference.

In one illustrative process for producing the absorbent articles, a core material can be produced by forming a web of material and coupling the formed web to a backing layer. In some embodiments, cellulose fiber comprising fluff pulp, e.g., dry wood pulp cardboards produced by means of a hammer mill, can be used to provide the core material. In one embodiment, a layer of fibers, e.g., typically present in irregular or random orientations, can be conveyed to a first pair of calender rollers on a strainer conveyer belt. One of the rollers can be heated or have a desired surface temperature, e.g., 200-220° C., while the other roller can remain unheated. If desired, the web can be moisturized, coated or sprayed with a desired substance prior to entering the gap between the two rollers. In some embodiments, the resultant moisture content of the material is about 5 to 10 percent by weight. During passage of the material between the rollers, the moisture content can be reduced from the use of the heated roller. After passage through the rollers, the cellulose fibers are compressed to provide a loose non-woven. The non-woven can be moisturized, coated or sprayed with a desired substance after exiting the rollers.

In certain embodiments, the loose non-woven can be provided to a pair of calender rolls. The loose non-woven can be subjected to an array of point-shaped pressure zones, where the irregularly arranged fibers are pressed onto each other under high pressure, such that a close fusion of the fiber bodies occurs and a fiber web with a desired pattern can be provided that generally will not separate after the pressure is released. The particular pattern is not critical and illustrative patterns include pinpoint patterns, square patterns, circular patterns and other geometric patterns. The pressure used can result in “melting” of the fiber materials and provide close bonding. For example, through focused high pressure and crowding of the fibers, the loose cellulose or pulp fibers can be bonded together in free spaces resulting in an overall very strong fiber web. The calendar rolls can be operated at room temperature, e.g., between 18-25° C., but one or more of them may also be heated if desired. In some examples, the pressure provided by the calender rolls can vary between 100 to 600 MPa, e.g., 500 MPa, but higher pressures can also be used. In certain examples, the resulting fiber web of material can comprise a basis weight, for example of about 50-1500 g/square meter. The fiber web exiting the calenders is significantly more tear resistant than the web entering the calender rolls. In some examples, the resulting fiber web may then be provided to a drawing roller, and, if desired, wrapped onto a take-up roller with the use of a driver roller.

In certain examples, the loose material provided to the first set of rollers can be an inexpensive mass material that is available in large amounts. For example, fluff pulp with a whiteness of 85 to 89% can be used, which in turn means that a significant lignin and residue content is still present, which can assist in improving the bonding behavior. The fiber length of the material can vary and is desirably long enough such that they bridge the distance between the pressure zones. The additives described herein can be used, for example, in various amounts. In some instances, fluff pulp can be supplemented with superabsorbers with 0.5 to 70 percent in weight, preferably 5 to 30 percent in weight, and thereafter sent through the high-pressure calender rolls. The superabsorbers generally have no bonding effect but become trapped in the non-woven once it is processed.

In certain embodiments, the core material can be produced without the use of any binding agents. By producing the core material without using any binding agents, the recyclability and compostability of the product is improved. In addition, the production becomes less expensive and is simpler because stations for applying and curing are not required. In other embodiments, if desired, the core material can be produced using one or more binding agents or binding agents may be used to couple the core material to the backing layer and/or top layers as described herein.

In some embodiments, the finished core material may comprise numerous irregular cellulose fibers that are coupled by fusion in the pressure zones. The material itself can comprise a high tear strength and, in addition, a high absorption capacity, which is increased even further through the use of superabsorbers such that it can be used as packaging material, for hygiene articles other articles described herein.

In certain examples, the finished core material can be glued, welded, laminated or otherwise coupled to a backing layer, film or sheet. The backing layer may be pre-coated with an adhesive and coupled to the core material using one or more additional roller pairs to press the core material against the backing layer. Depending on the type of adhesive used, ultraviolet light, visible light, heat or other stimulus may be applied to the resulting composite to assist in curing of the adhesive.

In some instances, the backing layer can be produced using one or more thermoplastics or may include one or more thermoplastics. Thermoplastics provide desirable attributes including enhanced flexibility, desirable weight-to-strength ratios and easy processing. Illustrative thermoplastic materials include, but are not limited to, a polyethylene, a polypropylene, a polybutylene terephthalate, a polyester-polypropylene, a polyethylene terephthalate, an acrylonitrile butadiene styrene, a cellulose acetate, an ethylene-vinyl acetate, an ethylene vinyl alcohol, a fluoroplastic such as polytetrafluoroethylene, a polyoxymethylene, a polyacrylate, a polyacrylonitrile, a polyamide, a polyamide-imide, a polyetheretherketone, a polyaryletherketone, a polybutadiene, a polybutylene, a polycaprolactone, a polychlorotrifluoroethylene, a polyetherketoneketone, a polycarbonate, a polycyclohexylene dimethylene terephthalate, a polyhydroxyalkanoate, a polyketone, a polyester, a polyetherimide, a polysulfone, a polyimide, a polylactic acid, a polymethypentene, a polyphenylene oxide, a polyphenylene sulfide, a polyphthalamide, a polystyrene, a polytrimethylene terephthalate, a polyurethane, a polyvinyl acetate, a polyvinyl chloride, a polyvinylidene chloride, a styrene-acrylonitrile, combinations thereof and other suitable thermoplastics. In some embodiments, the backing layer can comprise two or more different thermoplastics. In other embodiments, the backing layer can comprise three, four, five, six, seven, eight or more different thermoplastics. In certain embodiments, the backing layer can comprise at least one thermoset and at least one thermoplastic material. In certain embodiments, the backing layer can be a non-woven material. For example, a non-woven material produced using a polyester and a polyethylene can be used as the backing layer. In other configurations, a non-woven material produced using a polyethylene and a polypropylene can be used as the backing layer. In additional configurations, a non-woven material produced using a polyester and a polypropylene can be used as the backing layer. In certain configurations, at least one of a polyethylene, a polypropylene, and a polyester is used in combination with another thermoplastic material to provide a backing layer suitable for use in an article.

In certain examples, a single sheet of backing layer can be used to provide a plurality of independent absorbent articles that may be separated by perforations. For example, the backing layer can be coupled to the core material by pressing, rolling, using adhesives, using laser welding, melting or other processing steps as described herein. In some examples, a hot-melt adhesive can be placed between the backing layer and the core material and the resulting composite can be heated to couple the backing layer to the core material. If desired, the composite can be passed through rollers, placed into a press or mold or otherwise a desired amount of pressure can be applied using suitable methods such as those described in U.S. Pat. No. 6,675,702, for example. Once the adhesive has cured, the composite sheet can be perforated in desired areas such that an article or articles of a desired size may be produced by tearing or separation at the perforations. For example, in a typical operation a sheet comprising of 2, 4, 6, 8 or more lanes of individual and separate articles can be produced. In some examples, perforation lines can be present between lanes and/or between rows of the layers.

In certain configurations, additional layers, e.g., cover layers or other layers can also be present in the absorbent articles. For example, it may be desirable to include a top layer on the absorbent core layer to provide for additional functionality or for aesthetic reasons. In some examples, the top layer may comprise one or more thermosets or thermoplastics as described herein. For example, where the top layer comprises a thermoset, the thermoset may be, for example, a polyester-fiberglass system, vulcanized rubber, a phenolic resin, a phenol-formaldehyde resin, a urea-formaldehyde foam, a melamine resin, a melamine-formaldehyde resin, an epoxy resin, a polyimide, a cyanate ester, a polycyanurate, a polyester thermoset such as, for example, an unsaturated polyester that can be cross-linked and other suitable thermoset materials and combinations of thermoset materials. If desired, the thermoset can be used with one or more cross-linking agents to facilitate setting of the material during processing. In some examples, the top layer can comprise two or more different thermosets. In other embodiments, the top layer can comprise three, four, five, six, seven, eight or more different thermosets. Where the top layer comprises a thermoplastic, the thermoplastic may be, for example, a polyethylene, a polypropylene, a polybutylene terephthalate, a polyester-polypropylene, a polyethylene terephthalate, an acrylonitrile butadiene styrene, a cellulose acetate, an ethylene-vinyl acetate, an ethylene vinyl alcohol, a fluoroplastic such as polytetrafluoroethylene, a polyoxymethylene, a polyacrylate, a polyacrylonitrile, a polyamide, a polyamide-imide, a polyetheretherketone, a polyaryletherketone, a polybutadiene, a polybutylene, a polycaprolactone, a polychlorotrifluoroethylene, a polyetherketoneketone, a polycarbonate, a polycyclohexylene dimethylene terephthalate, a polyhydroxyalkanoate, a polyketone, a polyester, a polyetherimide, a polysulfone, a polyimide, a polylactic acid, a polymethypentene, a polyphenylene oxide, a polyphenylene sulfide, a polyphthalamide, a polystyrene, a polytrimethylene terephthalate, a polyurethane, a polyvinyl acetate, a polyvinyl chloride, a polyvinylidene chloride, a styrene-acrylonitrile, combinations thereof and other suitable thermoplastics. In some embodiments, the top layer can comprise two or more different thermoplastics. In other embodiments, the top layer can comprise three, four, five, six, seven, eight or more different thermoplastics. In certain embodiments, the top layer can comprise at least one thermoset material and at least one thermoplastic material. In certain instances, a cellulose based material such as tissue, pulp or the like can be placed over exposed areas of the absorbent article. In some embodiments, two or more different materials can be added to the exposed areas. The material can be added in the form of a sheet, strips, segments or the like.

In certain embodiments, the overall thickness of the individual articles can vary depending on the intended use of the article. In some embodiments, the articles may be designed to be thin, e.g., 2 mm or less, whereas in other examples it may be desirable to increase the overall thickness of the article to provide for increased absorption, for example. In some embodiments, the thickness of the article can vary from about 1 mm to about 20 mm, more particularly, from about 1.5 mm to about 10 mm, for example, about 1.5 mm to about 9 mm, about 1.75 mm to about 5 mm or any value within these illustrative ranges.

In certain embodiments, the overall dimensions, geometry and shape of the article can vary. In some embodiments, the article can take the form of an individual pad with a width of about 4 inches by about 6 inches, more particularly a width of about 3 inches to about 5 inches, e.g., about 2 inches by about 2 inches by about 3 inches. In certain configurations, each layer of the absorbent articles may be about 12 inches to about 120 inches long, more particularly about 36 inches to about 96 inches long. In certain configurations, each layer of the absorbent articles may be about 12 inches to about 96 inches wide, more particularly about 24 inches to about 72 inches wide. In certain configurations, the dividers used herein can be sized and arranged such that they generally minor the width and/or length of the layers of the stack, whereas in other examples, the divider can be sized greater than or less than the dimensions of the stacks.

In certain embodiments, the articles can be sterilized prior to, during or after packaging in the containers. Many different sterilization methods can be used and desirably, non-moisture based sterilization methods are used so that the core material is not unnecessarily exposed to moisture prior to use. Illustrative types of sterilization methods include, but are not limited to, gamma radiation, electron beam radiation, X-ray radiation, ultraviolet radiation, ozonation, ethylene oxide gas exposure and other suitable non-water based sterilization methods. In some examples, the articles may be packaged in plastic or paper bags or receptacles, sealed from the ambient and then sterilized using one or more suitable methods and materials. In other examples, sterilization may take place prior to packaging or immediately prior to use of the article.

In certain examples, the articles described herein can include cosmetic agents. For example, the core material, top layer (when present) or both can include one or more cosmetic agents designed to provide a desired effect. Illustrative cosmetic agents include, but are not limited to, a moisturizer, a perfume, a sunblock, an exfoliant, a lotion, a powder, a polish, a sanitizer, a salt, a butter, a skin lightener, an anti-acne agent, an anti-wrinkle agent, a tanning agent, an oil, or other suitable cosmetic agents commonly applied to the skin of humans. In some examples, the article can be used with a cosmetic agent once the cosmetic agent has been applied to the skin or other desired area. For example, the cosmetic agent can be applied to the skin and then an article can be applied over the same area of the skin. In other examples, the article can be first applied to the skin to remove any unwanted moisture and then a cosmetic agent may be applied optionally followed by application of another article. For example, it may be desirable to apply the article to burn patients to remove residual moisture from the areas prior to application of desired cosmetic or therapeutic agents.

It will be recognized by the person of ordinary skill in the art, given the benefit of this disclosure, that the articles described herein can be configured or sized and arranged in many different forms, shapes and arrangements. In one embodiment, the articles described herein can be sized and arranged for use as a meat pad, a cosmetic pad, a surgical pad, a nursing pad, a feminine hygiene product, a diaper, a helmet or hat insert or other uses. For example, the dimensions of the article may be similar to, or the same as, the packaging used to package meat products such as, for example, beef, pork, chicken, lamb or other meats. For example, each of the individual absorbent articles can be used to absorb fluids from surfaces to prevent growth of mold, bacteria or other organisms of the surfaces. For example, the articles can be placed in food packaging to absorb water or moisture within the package to prevent the food from being exposed to the water. In some examples, the articles may be placed in shipping crates, plastic bags or other forms of packaging commonly used to ship and/or sell food products such as produce, meats, cheeses or other foods.

In some embodiments, the articles described herein can be used as liners for shelves in pantries, refrigerators or other areas where it may be desirable to remove moisture. For example, sections of the article may be cut or the article may be sized and arranged to be inserted into crisper drawers or placed on refrigerators shelves to absorb excess moisture from food products in the refrigerator. In some examples, the article can be used with an insert designed to retain the article in place or the shelf or drawer may include fittings, tabs or holder to hold the article in place during use.

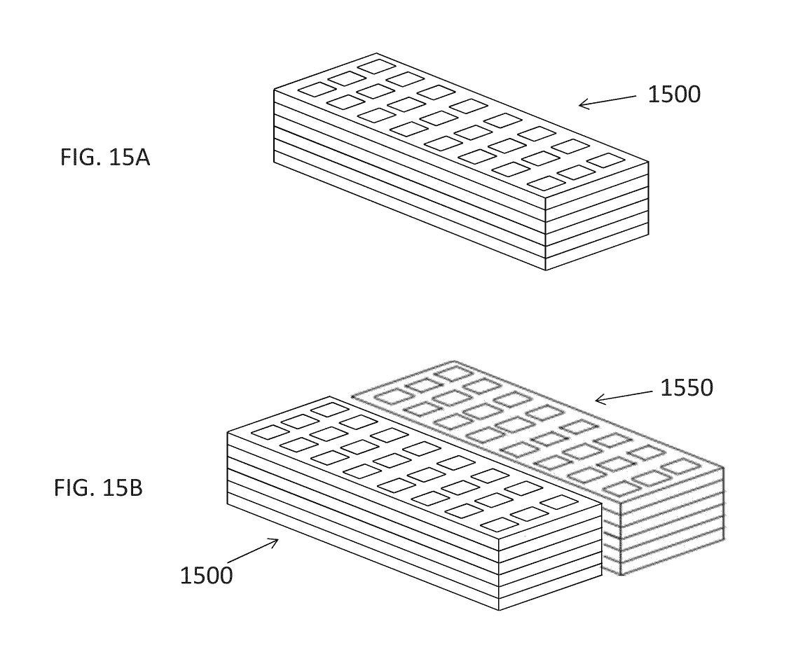

In certain examples and referring to FIG. 15A, a stack of layered articles is shown. While three lanes are shown in the stack 1500, less than three or more than three, e.g., four, five, six, etc., lanes may be present. Similarly, the exact number of rows presents in each layer may also vary. The stack 1500 comprises layers 1510 each comprising a plurality of absorbent articles. The backing layer of the absorbent articles generally forms each layer of the stacked layers. Perforations may exist between the rows and/or lanes of the stack 1500 to facilitate removal of the lanes and/or rows from the stack. In one use of the stack 1500, the left lane of absorbent articles can be stripped away from stack 1500 to leave the middle and right lanes. The backing layer can be cut or torn between rows to provide individual articles. The articles in the middle lane may then be removed and cut to provide individual articles and leaving the terminal or right lane of the stack. This lane can then be removed, and the articles can be separated from other articles to provide individual absorbent articles. An illustration showing two stacks is provided in FIG. 15B. A bottom layer of the stack 1500 can be coupled to a top layer of the stack 1550 to provide for continuous removal of lanes between the stacks 1510, 1550.

In placement of the stacks 1500, 1550 into a container, a temporary divider or insert can first be inserted during stacking of the layers. The temporary divider may not be thick enough or comprise a suitable material to provide any substantial support, but may assist in keeping the layers in stack 1500 from interleaving in the layers of stack 1550, which could lead to processing complications. In some instances, the temporary divider may be a flexible or bendable material such as paper, a plastic, a metal such as aluminum, a laminate or the like. In some instances, the height of the temporary divider is less than the height of the final stacks in the container. For example, the temporary divider can be inserted until about 50% of the final height of the stack and then it can be removed. In some instances, the temporary divider is removed and followed by placement of one of the dividers described herein, e.g., a U-shaped divider, a Z-shaped divider, an X-shaped divider, a C-shaped divider, a lattice shaped divider or other dividers. In other instances the temporary divider may remain in place and sit adjacent to the support divider or may be crushed or flattened by insertion of the support divider into the container.

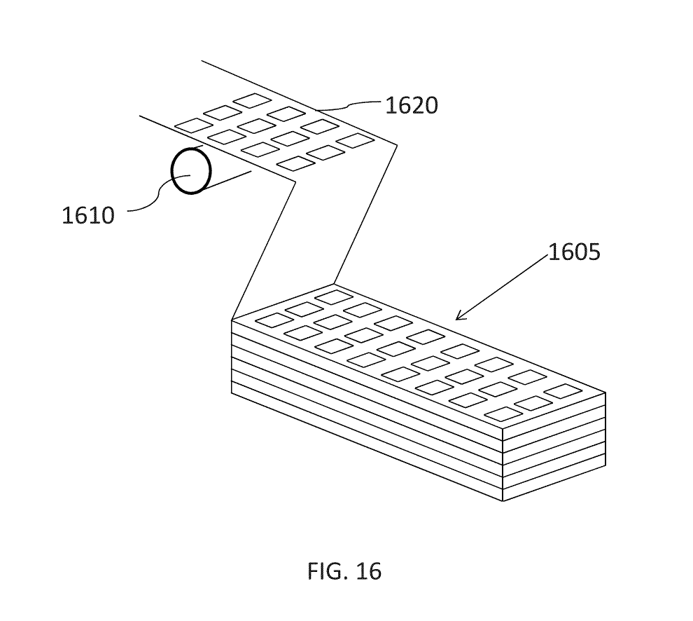

To produce the various layers of the articles, a continuous roll process can be used to form the articles on a backing layer, e.g., to form a plurality of individual articles on a common backing layer. The formed articles can be rolled over a feed roller that can move forward and backward to layer the articles in a container. In some instances, one or more press rollers or crimpers may be present to press the backing layer at fixed locations to provide a crease between layers. A generalized schematic of such a system is shown in FIG. 16. A feeding device 1610 rotates to feed the sheet 1620 of the plurality of absorbent articles into a stack 1605 in the general direction. If desired, the feeding device 1610 may move back and forth to assist in stacking of the sheet into the layers. In instances where a container comprises a first stack and a second stack of articles, the first stack can be provided followed by the second stack. In other instances, the stacks may be provided in a parallel manner by feeding two separate sheets over the feeding device and stacking each sheet into separate adjacent stacks. In some instances, the divider is inserted into the container prior to stacking of any articles into the container or a temporary divider can also be used as described herein. In other instances, some portion of the stack is provided in the container and then the support divider is inserted. In certain configurations, the entire first stack and the second stack are provided in the container, and then the divider is inserted into the container comprising the first stack and second stack. If desired, the container can be positioned on a support, e.g., a tray or moving table, to move the container back and forth rather than moving the feeding device 1610. In other instances, the support remains stationary during stacking of the articles.

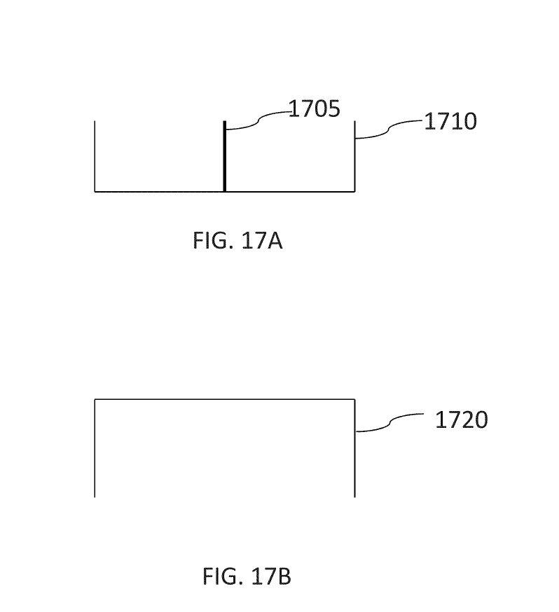

In certain embodiments, the containers used herein may comprise a bottom portion and a top portion. Referring to FIGS. 17A and 17B, a bottom portion 1710 generally is sized and arranged to receive two or more stacks. A divider 1705 is shown as inserted into the bottom portion 1710. A top portion 1720 is sized and arranged to slide over the stacks prior to shipping. The top portion can include flaps on an upper surface to permit removal of the stacks after the container has arrived at a facility. The height of the bottom portion 1710 is typically less than the height of the stack, though this height difference is not required. The top portion 1720, when mated to the bottom portion 1710, acts to seal the stacks within the assembled container. If desired, the entire container assembly can be wrapped in plastic or other materials to aid in sealing the stacks within the container. Straps or other materials can also be used to tie the top portion 1720 to the bottom portion 1710.

In certain configurations, the dividers described herein can permit loading of additional absorbent articles into the containers compared to a container lacking a divider. For example, a single container may comprise 20,000 or 30,000 or even 40,000 or more absorbent articles. The absorbent articles can be present in lanes with anywhere from 2-12 lanes, for example, of absorbent articles being present in any one layer. The dividers can be used in packaging technologies other than absorbent articles including, for example, table napkins, textiles, removable paper notes, and other similar applications. The exact length and width of the dividers can vary depending on the particular configuration of the divider, and illustrative lengths are from about 72 inches to about 120 inches, e.g., about 80 inches to about 108 inches or about 90 inches to about 100 inches long. Illustrative widths include, for example, about 24 inches to about 60 inches, more particularly about 32 inches to about 48 inches, e.g., about 36 to about 44 inches.

Certain specific examples of dividers are described in more detail below.

### Example 1

In one configuration, a divider is produced from corrugated cardboard (44 ECT strength) with a length of about 92 inches and a width (height) of about 40 inches. The cardboard is formed into a Z-shaped divider that generally bisects the width of the container.

### Example 2

In another configuration, a divider is produced from corrugated cardboard (44 ECT strength) with a length of about 92 inches and a width (height) of about 37 inches, which can be less than the overall height of a container comprising the stacked layers. The cardboard is formed into a Z-shaped divider that generally bisects the width of the container.

### Example 3

In one configuration, a divider is produced from corrugated cardboard (44 ECT strength) with a length of about 92 inches and a width (height) of about 40 inches. The cardboard is formed into a U-shaped divider.

### Example 4

In one configuration, a divider is produced from corrugated cardboard (44 ECT strength) with a length of about 92 inches and a width (height) of about 37 inches, which can be less than the overall height of a container comprising the stacked layers. The cardboard is formed into a U-shaped divider.

### Example 5

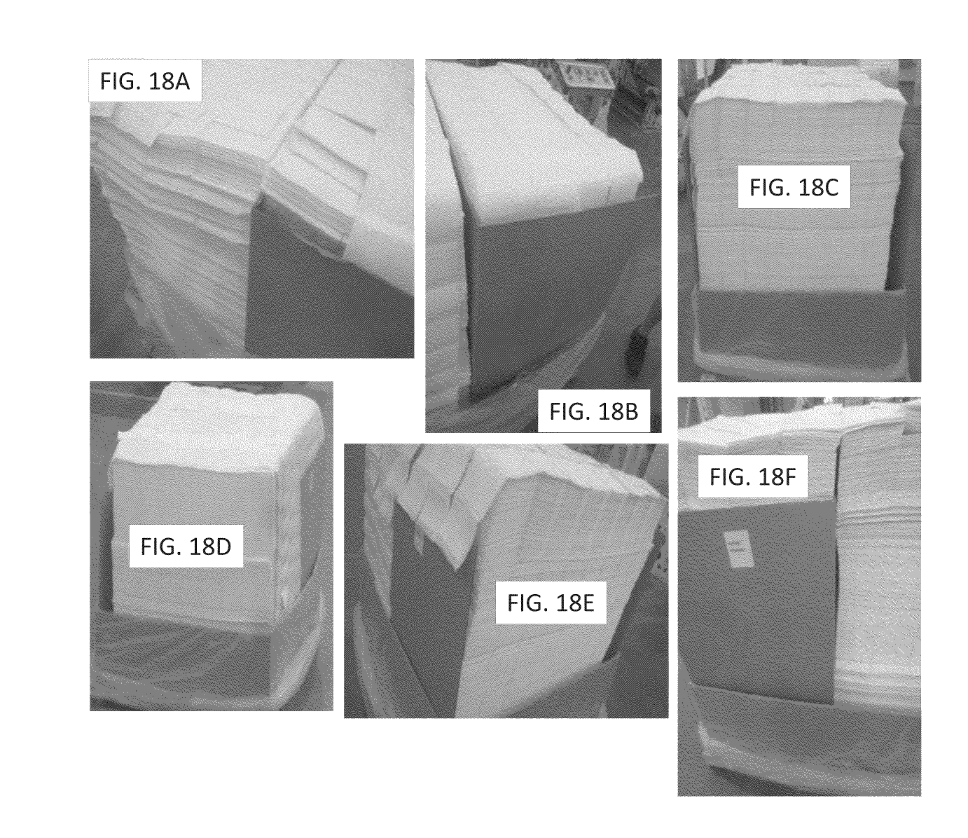

Referring to FIGS. 18A-18F, various photographs of a Z-shaped divider inserted between two stacks of a layered absorbent articles is shown. The Z-shaped divider has a height that is greater than 50% of the height of the stack and less than overall height of the stack. The Z-shaped divider comprises three different sections and is produced from corrugated cardboard.

### Example 6

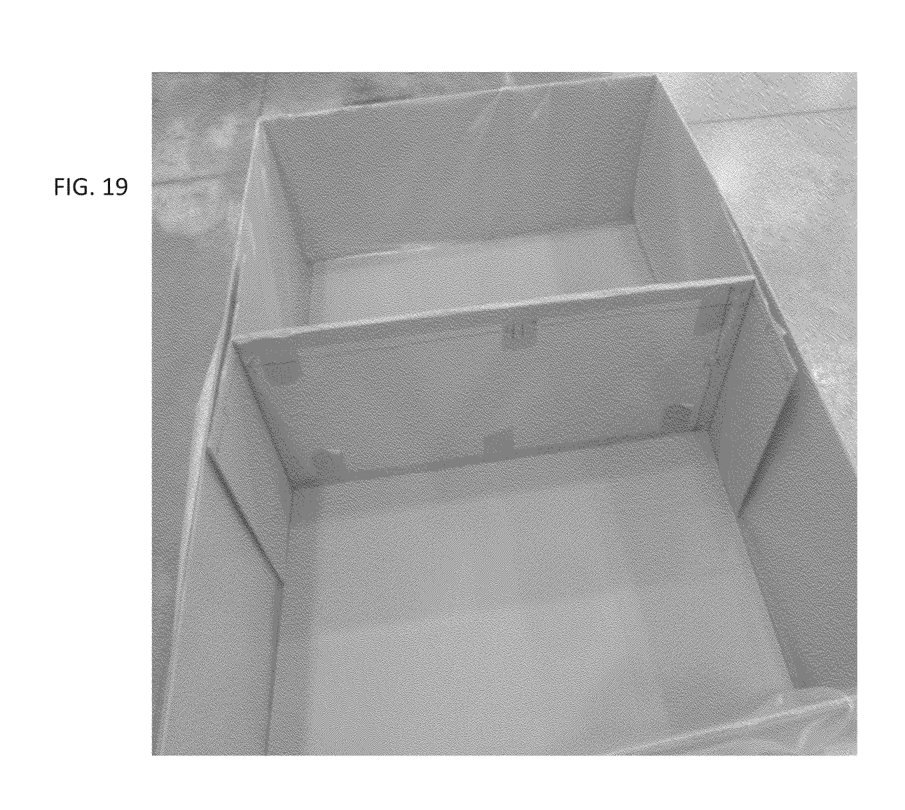

Referring to FIG. 19, a C-shaped divider is shown that has been inserted into a container prior to stacking of the layered stacks. The C-shaped divider can be configured as a temporary divider or as a support divider. In position, the divider roughly bisects the container and has a length for the central portion of about 41 inches with arms of the “C” having a length of about 8 inches.

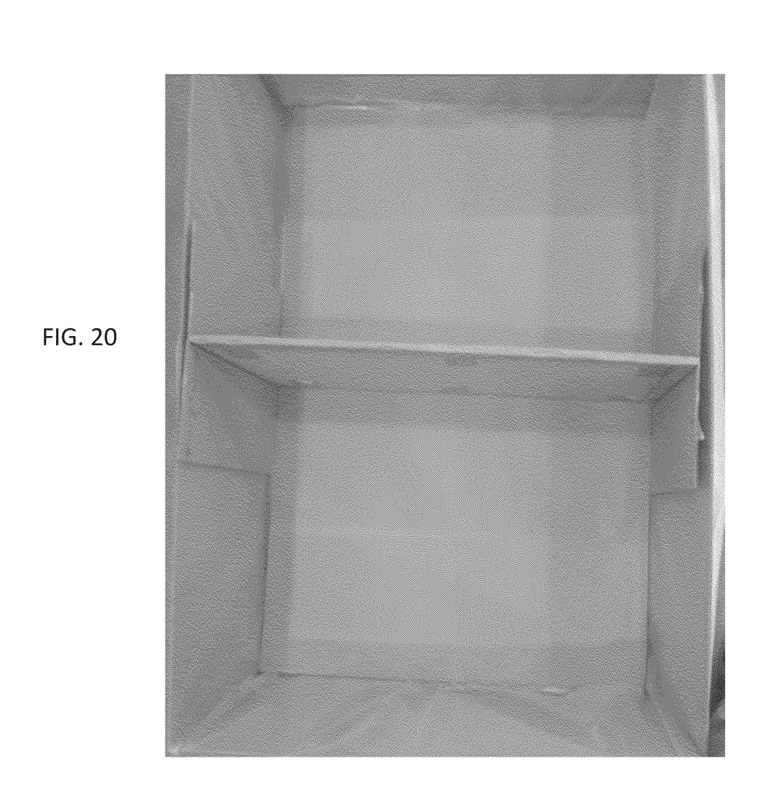

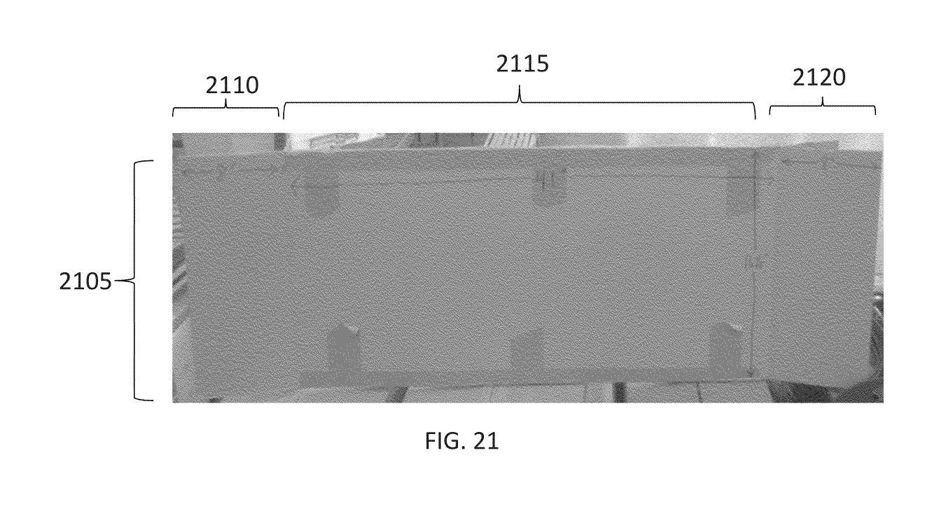

Referring to FIGS. 20 and 21, three separate sections 2110, 2115 and 2120 of the C-shaped divider are shown. The length of the sections 2110 and 2120 are each about 8 inches, and the length of the section 2115 is about 41 inches long. The height 2105 of the divider is about 16⅛ inches. As noted herein, however, the height, length and width of the divider may vary.

When introducing elements of the aspects, embodiments and examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

Although certain aspects, examples and embodiments have been described above, it will be recognized by the person of ordinary skill in the art, given the benefit of this disclosure, that additions, substitutions, modifications, and alterations of the disclosed illustrative aspects, examples and embodiments are possible.

1-100. (canceled)

101. A method of packaging a plurality of absorbent articles, the method comprising:
stacking a first stack comprising a plurality of absorbent articles in a container, in which the absorbent articles of the first stack are arranged in lanes in the first stack;
stacking a second stack adjacent to the stacked first stack in the container, the second stack comprising a plurality of absorbent articles, in which the absorbent articles of the second stack are arranged in lanes in the second stack; and
inserting a divider between the first stack and the second stack, in which the divider is sized and arranged to provide support to at least one lane of absorbent articles of the first stack.

102. The method of claim 101, further comprising inserting the divider into the container after the first stack is stacked and before the second stack is fully stacked.

103. The method of claim 101, further comprising inserting the divider into the container before either of the first stack and the second stack are fully stacked.

104. The method of claim 101, further comprising configuring the divider to comprise a U-shaped member, a Z-shaped member, a C-shaped member, a lattice member or an X-shaped member.

105. The method of claim 101, further comprising coupling a bottom layer of the first stack to a top layer of the second stack.

106. The method of claim 101, further comprising coupling a bottom layer of the first stack to a bottom layer of the second stack.

107. The method of claim 101, further comprising inserting a first divider and a second divider between the first stack and the second stack.

108. The method of claim 101, further comprising configuring each of the first stack and the second stack with at least 4 lanes of absorbent articles per layer.

109. The method of claim 101, further comprising covering the container with another container after insertion of the divider.

110. The method of claim 101, further comprising covering the container with another container comprising the divider.

111. A method of providing support to a stack of absorbent articles, the method comprising inserting a divider between a first stack of absorbent articles and a second stack of absorbent articles each positioned in a container, in which each of the first stack and the second stack comprises a plurality of individual absorbent articles, in which the absorbent articles of the first stack are present in stacked layers with each layer of the first stack comprising a plurality of the individual, absorbent articles arranged in lanes.

112. The method of claim 111, further comprising inserting the divider into the container after the first stack is stacked and before the second stack is fully stacked.

113. The method of claim 111, further comprising inserting the divider into the container before either of the first stack and the second stack are fully stacked.

114. The method of claim 111, further comprising configuring the divider to comprise a U-shaped member, a Z-shaped member, a C-shaped member, a lattice member or an X-shaped member.

115. The method of claim 111, further comprising coupling a bottom layer of the first stack to a top layer of the second stack.

116. The method of claim 111, further comprising coupling a bottom layer of the first stack to a bottom layer of the second stack.

117. The method of claim 111, further comprising inserting a first divider and a second divider between the first stack and the second stack.

118. The method of claim 111, further comprising configuring each of the first stack and the second stack with at least four lanes of absorbent articles per layer.

119. The method of claim 111, further comprising covering the container with another container after insertion of the divider.

120. The method of claim 111, further comprising covering the container with another container comprising the divider.

121-142. (canceled)
